(19) United States
(12) Patent Application Publication
(10) Pub. No.: US 2025/0046393 A9
(43) Pub. Date: Feb. 6, 2025

(54) METHODS FOR RANKING AND/OR SELECTING TUMOR-SPECIFIC NEOANTIGENS

(71) Applicant: CECAVA GMBH & Co. KG, Tubingen (DE)

(72) Inventors: Saskia Biskup, Tubingen (DE); Florian Battke, Tubingen (DE); Dirk Hadaschik, Tubingen (DE); Christina Kyzirakos-Feger, Tubingen (DE); Simone Kayser, Tubingen (DE); Moritz Menzel, Tubingen (DE); Sorin Armeanu-Ebinger, Kusterdingen (DE); Magdalena Feldhahn, Kusterdingen (DE); Dirk Biskup, Tubingen (DE)

(21) Appl. No.: 17/271,665

(22) PCT Filed: Aug. 28, 2019
(86) PCT No.: PCT/EP19/73025

§ 371 Date: Feb. 26, 2021

Related U.S. Application Data
(63) Continuation of application No. 16/114,878, filed on Aug. 28, 2018, now abandoned.

(30) Foreign Application Priority Data
Aug. 28, 2018 (EP) 18191148.8

(51) Int. Cl. G16B 20/20; G01N 33/574; G16H 50/20

(52) U.S. Cl. CPC G16B 20/20; A61K 39/0011; G01N 33/574; G16H 50/20

(57) ABSTRACT

The present invention relates to the ranking/selection of tumor-specific neoantigens of a subject having cancer. The present invention also provides methods using the ranked/selected tumor-specific neoantigens in. for example. the treatment or prevention of cancer. Ranked and selected neoantigens may be used as biomarkers in the diagnosis, monitoring and/or prognosis of tumor diseases.

The present invention relates to the ranking/selection of tumor-specific neoantigens of a subject having cancer. The present invention also provides methods using the ranked/selected tumor-specific neoantigens in, for example, the treatment or prevention of cancer. Ranked and selected neoantigens may be used as biomarkers in the diagnosis, monitoring and/or prognosis of tumor diseases.

Within the past decade fresh enthusiasm has revived around the possibility of using vaccines as anticancer agents. Data collected by dedicated translational researchers document that a variety of anticancer vaccines, including cell-based, DNA-based, and purified component-based vaccines, are capable of circumventing the poorly immunogenic and highly immunosuppressive nature of tumors and elicit therapeutically relevant immune responses in cancer. Due to observed antitumoral T cell answers induced by tumors, “off-the-shelf” peptide vaccines (targeting mainly unmutated tumor associated antigens like in KRAS, Gastrin G17DT, HSP-CC-96, WT1, VEGF-R and2, hTERT, Her2/neu, KIF20A), recombinant vaccines (MUC-1 and CEA in poxvirus with GM-CSF), live attenuated Listeria Mesothelin-expressing vaccines, irradiated whole allogenic tumor and Listeria and whole inactivated tumor cell vaccines (Algenpantucel-L, Allogeneic GM-CSF) have been evaluated for therapy in cancer.

These studies have generated promising results yet failed in inducing robust, statistically relevant improvement in patient survival. Nevertheless they identified several critical aspects for the design of successful next generation cancer vaccines, namely: cancer vaccines should be tumor specific and distinct from self-proteins, the applied adjuvant should potently activate antigen-presenting cells to stimulate an antigen specific cytotoxic T lymphocyte (CTL) and T helper lymphocyte mediated immune response and strategies for breaking immunological tolerance should be included.

Non-self antigens like unique neoantigens created by mutations in a tumor's genome have hitherto been cumbersome to detect. The search including cDNA expression cloning, serologic analysis of recombinant cDNA expression libraries (SEREX), and reverse immunological approaches has become dramatically simplified with the advent of NGS technology. Entire cancer exomes can be sequenced and compared with normal exome in order to reliably identify the tumor specific and highly individual mutations. Subsequently, bioinformatic algorithms can be applied to predict which mutation-derived, altered protein sequences may give rise to new antigens (neoantigens), which can be presented as peptides via the patient individual HLA molecules on the surface of the respective tumor cells. By integrating the identified neoantigens into cancer vaccines now provides the fundamental new opportunity to target specifically the patient individual tumor aberrancies. Such a personalized approach integrates the tremendous heterogeneity of tumors of same tissue origin in different individuals and increases the potential to elicit a powerful anti-tumor immune response, since T cells recognizing neo-antigens with high affinity have not been eradicated by thymic negative selection. Besides of driver mutations therapeutically useful targets may also be generated from individual passenger mutations giving rise to highly immunogenic neoantigens.

Considerable progress towards significant efficacy has been obtained by combining anticancer vaccines with a relatively varied panel of therapies, which help break the immune suppressive nature of the tumor milieu. These include diverse inhibitors of immune checkpoints, targeted therapies and/or chemotherapeutics (i.e. oxaliplatin) that can provoke immunogenic cell death (ICD).

From WO 2017/205823A1, methods and systems for personalized genetic testing of a subject are known, where a sequencing assay is performed on a biological sample from the subject, which then leads to genetic information related to the subject. It is suggested that nucleic acid molecules are array-synthesized or selected based on the genetic information derived from data of the sequencing assay. At least some of the nucleic acid molecules shall then be used in an assay which may provide additional information on one or more biological samples from the subject or a biological relative of the subject.

A method for the identification of neoantigens is provided in WO 2017/011660, which uses whole exome sequencing and various functional criteria. A final priority score is determined based on the results obtained for selected criteria, subsequent to excluding neoantigens below specific threshold values for individual criteria. Further, WO 2018/045249 provides a method for the identification of cancer-specific immunogenic peptides in a cross-species manner, in particular in mouse and human cancer cells. The method ranks peptides according to various criteria using score values.

However, while genetic information and functional data may help in personalizing medical treatment, a large number of problems remain to be solved.

First of all, as with any measurement, the genetic information derived from a person's biological samples may be incorrect to a certain extent, e.g. because the information contains a certain amount of errors. Drawing conclusions from such genetic information is difficult or even impossible given that at the time of this invention, medical knowledge still is limited. For example, some rare forms of tumors and cancer may exist that as of yet cannot be attributed with a sufficiently high degree of certainty to specific genetic information. Accordingly, even where a large wealth of genetic data relating to certain diseases exist, for example in the form of libraries, the best information included in such libraries may at one given time be different from the best information included in a similar library at a later time simply because an existing library of genetic data needs to be modified in view of scientific progress.

Then, both any library including medical data and the genetic information obtained from samples of a patient can be rather extensive so that comparing the genetic information obtained from a patient sample to data in one or more libraries can be very computationally intensive.

Also, where it is determined that certain neoantigens might be of particular relevance in view of a cancerous disease a patient suffers from or is believed to suffer from according to the best medical diagnosis available, the selection of neoantigens for therapeutic intervention will depend on which properties the neoantigens have. Such properties might for example be determined in-silico, that is by way of numerical calculation in view of certain assumptions as to their structure. However, the numerical calculations will be neither fully exact nor will the assumptions underlying the calculations or the structure assumed be fully correct. Even if experimentally validated functional data is included, the relevance of such data may not be correctly reflected during the selection process.

Nonetheless, despite errors, lack of knowledge, uncertainties and depending on the medical condition of a patients, in certain cases an effective treatment needs to be found both fast and at a cost that is acceptable.

In view of this, there is a need in the art to provide improved methods for ranking personalized neoantigens and uses thereof.

It is thus an object of the invention to inter alia provide novel and inventive methods for ranking personalized neoantigens.

The present invention thus provides a ranking and/or selection method for ranking and/or selection of neoantigens of a subject having cancer, wherein a plurality of potential neoantigens carrying at least one mutation considered to be cancer-specific is ranked/selected by the steps that
(a) for the subject having cancer a library of potential neoantigens is provided;
(b) for each of a plurality of potential neoantigens from the library, which plurality comprises at least four potential neoantigens, at least two descriptors are determined selected from
   (i) an indicative descriptor indicating whether the neoantigen is known to reside within a cancer-related gene or whether the neoantigen is not known to reside within a cancer-related gene;
   (ii) a classifying descriptor relating to the binning of a value indicative for the allele frequency of the at least one tumor-specific mutation in the neoantigen of the subject into one of at least three different classes ordered according to the intervals of values binned into each class;
   (iii) a classifying descriptor relating to the binning of a value indicative for a relative expression rate of the at least one variant within a neoantigen in one or more cancerous cells of the subject into one of at least three different classes ordered according to the intervals of values binned into each class;
   (iv) a classifying descriptor relating to the binning of a value indicative for a binding affinity of a neoantigen to a particular HLA allele present according to the subject's HLA type, into one of at least three different classes ordered according to the intervals of values binned into each class;
   (v) a classifying descriptor relating to the binning of a value indicative for a relative HLA binding affinity of the subject specific potential neoantigen as compared to the corresponding non-mutated wild-type sequence into one of at least three different classes ordered according to the intervals of values binned into each class;
   (vi) a classifying descriptor relating to the binning of a value indicative for a binding affinity to more than one HLA allele present according to the subject's HLA type, into one of at least three different classes ordered according to the intervals of values binned into each class;
   (vii) a classifying descriptor relating to the binning of a value indicative for the HLA promiscuity of a neoantigen into one of at least three different classes ordered according to the intervals of values binned into each class;
   (viii) a classifying descriptor relating to the binning of a value indicative for the reliability of predicting binding of the subject specific potential neoantigen to a HLA allele of the respective patient into one of at least three different classes ordered according to the intervals of values binned into each class;
   the determination of at least one of the at least two descriptors being such that the number of different classes into which the respective values are binned is smaller than the number of the potential neoantigens of the plurality;
(c) a combined score for each of the plurality of the potential neoantigens is calculated based on the at least two descriptors in a manner weighted such that the maximum possible contribution of at least one descriptor to the combined score will be lower than the maximum possible contribution to the combined score of at least one other descriptor;
(d) a ranking of the plurality of at least four potential neoantigens based on the combined scores is obtained.

The present invention furthermore provides a selection method for cancer-specific neoantigens personalized for treating an individual subject having cancer, wherein from a plurality of potential neoantigens carrying at least one mutation considered to be cancer-specific a selection is made by the steps that for the individual subject having cancer an individual library of potential neoantigens is provided; for each of a plurality of at least four potential neoantigens in the library at least two of an indicative descriptor indicating whether the neoantigen is known to reside within a cancer-related gene or whether the neoantigen is not known to reside within a cancer-related gene; a classifying descriptor relating to the binning of a value indicative for the allele frequency of the at least one tumor-specific mutation in the neoantigen of the subject into one of at least three different classes ordered according to the intervals of values binned into each class; a classifying descriptor relating to the binning of a value indicative for a relative expression rate of the at least one variant within a neoantigen in one or more cancerous cells of the subject into one of at least three different classes ordered according to the intervals of values binned into each class; a classifying descriptor relating to the binning of a value indicative for a binding affinity of a neoantigen to a particular HLA allele present according to the subject's HLA type, into one of at least two different classes ordered according to the intervals of values binned into each class; a classifying descriptor relating to the binning of a value indicative for a relative HLA binding affinity of the subject specific potential neoantigen as compared to the corresponding non-mutated wild-type sequence into one of at least three different classes ordered according to the intervals of values binned into each class; a classifying descriptor relating to the binning of a value indicative for a binding affinity to more than one HLA allele present according to the subject's HLA type, into one of at least three different classes ordered according to the intervals of values binned into each class; a classifying descriptor relating to the binning of a value indicative for the HLA promiscuity of a neoantigen into one of at least three different classes ordered according to the intervals of values binned into each class; a classifying descriptor relating to the binning of a value indicative for the reliability of predicting binding of the subject specific potential neoantigen to a HLA allele of the respective patient into one of at least three different classes ordered according to the intervals of values binned into each class; are determined such that for at least some of the values, the number of different classes, that the classifying descriptor bins the respective values into, is smaller than the number of the potential neoantigens; a combined score for each of the plurality of the potential neoantigens is determined based on the at least two descriptors and in a manner weighted such that the maximum possible contribution of at least one descriptor to the combined score will be lower than the maximum possible contribution to the combined score of at least one other descriptor; and ranking the plurality of at least four potential neoantigens based on the combined scores is determined; and a selection of at least one neoantigen and less than all neoantigens from the plurality of potential neoantigens in response to the ranking is made.

Accordingly, the present invention relates to improved methods for ranking/selecting cancer-specific neoantigens in a personalized manner. Methods of the prior art, such as methods provided in WO 2017/011660, comprise the exclusion of candidate peptides based on pre-determined threshold values. Once a peptide is excluded, it does not form part of the totality of candidate peptides for subsequent testing, even if the threshold value was nearly reached. Other methods, such as those provided in WO 2018/045249, comprise sorting of peptides according to results of functional testing. Sorting comprises the attribution of numerical values in a linear and equalized manner.

The inventors have made several surprising findings to arrive at the present invention. First, candidate peptides that have been considered non-functional according to one or more functional parameters using methods of the prior art may show surprisingly good functionality in subsequent testing. Thus, exclusion of peptides as it is part in methods of the prior art will result in a biased ranking order of candidate peptides. Second, a linear and equalized ranking such as in WO 2018/045249 inherently introduces a selection bias, considering that individual parameters will show a non-uniform contribution to factual effectiveness.

In contrast and as discussed further below and as shown in the appended examples, the methods of the present invention reduce selection bias by binning candidate peptides and attributing non-linear score values to bins and parameter contribution to the final score.

In the above disclosure of a method according to the present invention, reference has been made to the execution of several steps and the derivation and use of certain entities by using expressions such as indicative descriptors, indicative values, classifying descriptors, binning, classes, classes ordered according to the intervals of values, weighting, contributing and so forth. Furthermore, reference will also be made in the following description and appended claims to handicapping, filtering and so forth.

While it is believed that some or most of these common expressions will easily be understood by a person skilled in the art, non-limiting explanations are provided herein below.

In the present invention, reference is made to both an indicative descriptor and to classifying descriptors. The term “descriptor” is used having in mind a standard definition of a so-called molecular descriptor which sometimes is considered a final result of a procedure which transforms chemical information encoded within a symbolic representation of a molecule into a useful number or the result of some standardized experiment. For a specific substance, such a number might e.g. be a binding length within a molecule, a boiling point, the number of carbon atoms and so forth. However, here, when looking at the term “useful number” emphasis in the present application is not on “number” but on “useful”.

More precisely, the indicative or classifying descriptors in the present case need not necessarily be a numerical value but could also be e.g. alphanumerical information.

Regarding the term indicative descriptor indicating whether the neoantigen is known to reside within a cancer-related gene or whether the neoantigen is not known to reside within a cancer-related gene: Frequently, there is knowledge about whether or not a specific neoantigen is known to reside within a cancer-related gene or whether the neoantigen is not known to reside within a cancer-related gene. For example, the skilled person is aware that certain mutations exist which are considered driver mutations, passenger mutations and/or that are related to drug resistance. A “driver mutation” is a mutation that gives a selective advantage to a clone in its microenvironment, through either increasing its survival or reproduction. Driver mutations tend to cause clonal expansions. A “passenger mutation” is a mutation that has no known effect on the fitness of a clone but may be associated with a clonal expansion because it occurs in the same genome with a driver mutation. This is known as a hitchhiker in evolutionary biology. Within the present invention, a neoantigen is classified as residing within a cancer-related gene if it is determined to comprise a driver mutation, a mutation related to drug-resistance or a passenger mutation in known cancer-related genes. If, a neoantigen is known to reside within a cancer-related gene, the sentence “Yes, the neoantigen resides within a cancer-related gene”, would be an indicative descriptor, whereas a descriptor indicating that neoantigen is not known to reside within a cancer-related gene would be the clear-text sentence “No, the neoantigen is not known to reside within a cancer-related gene”. Obviously, shorter or other descriptors could be used. As non-limiting examples, the pair “Yes” and “No” would serve the exact same purpose, a pair of “Y”/“N”, “Ja”/“Nein”, “J”/“N”, “Oui”/“Non”, “O”/“N” or “A”/“B”, a pair of logical flags indicating a logical “0” or “1” etc. Also, instead of an alphanumerical indicative descriptor such as “Y” and “N”, numerical values could be used; e.g. a value larger than zero for YES and a value smaller than or equal to zero for NO. While using “0” and “1” would be a standard approach in this case, other values such as “0.0543” and “−7.231” could be used as long as they can be clearly distinguished from each other. In particular, a numerical value within a given range of values could be used, for example a value between 0 and 1. This can give additional advantages in certain instances. Suppose the indicative descriptor would be identical to 1 in case the there is a 6 sigma scientific certainty that a given neoantigen is known to reside within a cancer-related gene; while a value of “0.95” shall indicate that only a 5 sigma certainty exists that a given neoantigen is known to reside within a cancer-related gene etc. while a value of 0.5 shall indicate in this specific case that there currently is no scientific reason at all to assume that a given neoantigen is cancer-related. Here, the indicative descriptor while indicative might also provide additional information.

In the same way, classifying descriptors need not be numerical values either. This can easily be understood as well, and will be explained with respect to the physical size of person as the size is a more commonplace quantity than e.g. a relative HLA binding affinity. Suppose the person is a 6 years old girl that has a physical size of “127 cm” corresponding to “4 Feet 2 inches” which both are values indicative for the physical size of the person. If the unit used (cm, m, feet) is known, the size can be indicated as “4-2, “1.27”, “127”, “6-4” etc. Now, to a person not having regular contact with kids, this absolute value will not help to decide whether the girl is rather large for her age or not. However, as the physical size is generally determined and known for a large number of girls, the specific size (127 cm) can easily be compared to the size other girls of the same age have. It can thus be established that about 95% of girls having the same age are smaller. If only three classes are considered, for example small-medium-large, the specific 6 years old girl would most certainly be considered a “large” girl. The classifying descriptor in that case would be “large” but again could also be one of “S”, “M”, “L” or one of “1”, “2”, or “3” and so forth.

It is important to note that in the example, reference has been made to the size other girls have. In practice it can be determined whether e.g. a specific child is among the smallest 10% of its peer group (peer group=same age, same sex), among the largest 10% of its peer group or somewhere in between. (For the sake of completeness: The smallest 10% of 6 years old girls have a size up to 110 cm; the largest 10% have a size of at least 124 cm). Assigning the size of the child to a specific interval of ranges, (e.g. 0 cm-110 cm; 111 cm-123 cm; >124 cm) is referred to as binning. So, in order to determine that a 6 years old girl is a large girl, what is done is that a value indicative for the physical size of the girl is established (“127 cm”), the size is roughly compared with other girls by binning (“belongs to the largest 10%”) and a classifying descriptor is determined (“this is a large girl” or “L” or “3”) that relates to the binning of a value indicative for the physical size within a peer group.

Note that in the example the bins or intervals do not need to have the same size. A girl within the medium range as defined will not differ by more than 12 cm from another girl also having medium size. In contrast, a very small girl could be even smaller than 95 cm, so the maximum size difference within the “small bin” (or interval size of the bins) is not the same as in the “medium” bin. It should also be noted that for considering different aspects, different bin sizes can be used. For example, when determining whether a kid should have a somewhat higher or lower chair in school, other limits should be set than when deciding whether in view of a non-average size, medical treatment due to dysfunctions is indicated.

Basically, the same holds true for quantities other than physical sizes such as an allele frequency of the tumor-specific mutation in the neoantigen; a relative expression rate inside the tumor of the neoantigen-residing variant; a binding affinity of a neoantigen to a particular HLA allele present according to the subject's HLA type; a relative HLA binding affinity of the subject specific potential neoantigen as compared to the corresponding non-mutated wild-type sequence; the HLA promiscuity of a neoantigen. Here, also, numerical values can be calculated.

The numbers and units to describe such quantities may vary, but it will be obvious to the skilled person how for example, in a manner commonly known, e.g. a binding affinity can be determined. From such standard procedures commonly known, some (numerical) value will be determined e.g. for both the HLA binding affinity of the subject specific potential neoantigen and for HLA binding affinity of the corresponding non-mutated wild-type sequence. Then, when comparing the HLA binding affinity of the subject specific potential neoantigen determined in a manner commonly known to the HLA binding affinity of the corresponding non-mutated wild-type sequence, it could be determined whether the HLA binding affinity of the corresponding non-mutated wild-type sequence wild-type is equal to the HLA binding affinity of the subject specific potential neoantigen or is larger than that or is smaller than that. A corresponding value attributed could e.g. be “+1”, “0” or “−1”. It will be understood that all binding affinities are positive numbers so when establishing a relation such as “smaller than” or “equal to”, a ratio could just as well be determined and it could be checked whether this ratio is larger than 1, smaller than 1 or equal to one. So, a ratio could be determined as such indicative value, a percentage could be determined by multiplying the ratio by 100, a ratio of the squares could be determined as indicative value etc.

Regarding classes, several classes or number of binning ranges can be defined. In the example above, the size of a child was stated to be small, medium or large and it has already been stated that different ranges might be useful for different purposes. Also, for some purposes, it might be necessary to establish a different number of classes (such as XS, S, M, L, XL, XXL for absolute sizes when referring to clothing). In the same manner, the number of classes or ranges may differ from 3 for the quantities considered. However, using a number of ranges that is smaller than the number of elements in a sample examined is essential when differences between sample elements are to be regarded as irrelevant. By using a number of ranges smaller than the number of samples, at least two samples will fall into the same range and hence their absolute difference can be disregarded.

With respect to determining a combined score for each of the plurality of the potential neoantigens based on the at least two descriptors, such a combined score of a neoantigen can easily be obtained e.g. by adding certain values; the most simple approach would be to assign each descriptor to a specific numerical value and then add all the values for each neoantigen. (For example, where the descriptor relates to one of the three sizes S M and L, the numbers could be “1”, “2” and “3”).

However, according to the present invention, the scores are not simply added, but are combined in a specifically weighted manner. Basically, a weighted combination is well known, e.g. from a student of having a main subject of bioinformatics and several subsidiary subjects such as biochemistry. The credit points obtained in different courses usually will be weighted depending on whether or not the course was relating to a subsidiary subject or a main subject of the student, e.g. by multiplying courses in the main subject by a factor of two, that is, by assigning a weight of two. Note that the weights in the present invention are not simply combined in a weighted manner but in a specific manner such that the maximum possible contribution of at least one descriptor to the combined score will be lower than the maximum possible contribution to the combined score of at least one other descriptor. Also, it should be noted that while a simple addition of values certainly is resulting in a combined score, other ways of combining are possible, e.g. adding squared values or multiplying the values etc.

It is noted that in the above general description of the invention reference has been made to selecting at least two descriptors from the plurality of descriptors. It will be understood that for each neoantigen that is considered and ranked, the same descriptors are evaluated and used. Furthermore, it is noted that more than two descriptors can be selected. It is also possible that more than three or more than four or more than five descriptors are selected to obtain the ranking from a combined score and again, for all potential neoantigens, the same descriptors will be evaluated and used. Furthermore, it is possible to use all descriptors indicated to obtain a ranking and it would even be possible to use additional, unlisted descriptors that might also contribute in a similar manner to the overall score in a weighted manner to obtain the ranking.

The present inventors have surprisingly and unexpectedly found that the suggested combination of multiple determinations relating to antigen presentation on the surface of tumor cells of a subject in a manner allowing improved ranking/selection by a suitable combination of results thus provides patient-individualized tumor vaccines with improved characteristics over the use of prior art neoantigen prediction and ranking/selection methods. This finding is based on the surprising and unexpected results demonstrated in the appended examples. Therein, the effect of personalized neoantigen-based vaccines developed by the methods of the invention is shown (Example 6). Specifically, for a total of 12 patients with various malignancies long-term follow-up data is made available in the appended examples. The data surprisingly and unexpectedly demonstrates that the methods of the present invention can be used to uncover personalized neoantigens resulting in efficient neoantigen-specific T cell immune responses (CD4+ and CD8+).

Accordingly, a clear improvement over existing therapy can be achieved based on neoantigens ranked/selected according to the methods of the present invention. These methods thus provide a surprising and unexpected advantage of resulting vaccines due to the combination of multiple, at least two predictors and determinants and the subsequent combination of results, preferably in a weighted manner.

It has been concluded that surprisingly an improved prediction, ranking and selection can be obtained despite the lack of exact knowledge resulting from underlying imprecise or faulty measurements, rounding errors of in-silico calculations etc, if descriptors are binned into one of a few ranges. It is believed that in this way, while the small differences between descriptors will be disregarded most times, their overall value may still be coarsely taken into account without overestimating small, but-given factual precision-probably insignificant differences. For example, it is possible to distinguish values that indicate that the respective descriptor points to a negligible influence, to an influence that albeit small still is considered to be real, or to an influence that is considered to be very large.

Specifying one of these classes does not require that the respective value of the descriptor be determined with the highest precision possible. Rather, the errors that the values determined may show will be evened out by the classification. At the same time, by assigning a different weight to the descriptor depending on the range it is classified into, it also is taken into account that a very small value may bear an uncertainty larger than a higher value. Therefore, assigning a particularly low weight or score contribution to an otherwise important factor due to a low value reduces the noise otherwise associated with the low value. It shall be noted that by taking into account a plurality of descriptors, even where the value of one of the descriptors is close to the border of a range, minute errors may average out.

It should also be noted that even where certain parameters or values are determined in-silico, these determinations may still be dependent on initial physical measurements that as such are error-prone. For example, where an HLA-binding affinity is determined for a neoantigen, while such determination will depend on assumptions made based e.g. on a molecular structure predicted, the assumptions will still rely on some prior kinetic or other measurement. For example, the binding affinity of a neoantigen for a specific HLA molecule may be determined based on available data bases (e.g. IEDB) including e.g. results of in vitro binding assays of unrelated peptides shown to bind the respective HLA allele with a certain affinity. Compiling affinities of many peptides binding and not binding to a certain HLA allele allows to deduce peptide binding motives for the respective and related HLA alleles. Therefore such data bases allow calculations based on known properties of certain molecules or functional groups and predicted respective stereo-chemical structures, but into these data bases data will have been fed from physico-chemical experiments. Thus, in-silico determination of values will not be inherently error-free.

The results achievable demonstrate the superior characteristics of the method used to identify the employed peptides. These methods comprise in a preferred embodiment the combined use of at least several of the following parameters: origin from known cancer-related genes; allele frequency of at least one tumor-specific mutation in the neoantigen of the subject; relative expression rate of such neoantigen-residing variants in a cancerous cell of the subject; binding affinity to a particular HLA allele present according to the subject's HLA type; relative HLA binding affinity of the neoantigen as compared to the corresponding non-mutated wild-type sequence; binding affinity to more than one HLA allele present according to the subject's HLA type; HLA promiscuity of a neoantigen, wherein each neoantigen is categorized and each category is given a value, said value can be high if the neoantigen originates from a cancer-related gene; can increase with the variant allele frequency; can increase with the respective variant expression rate; can increase with the HLA binding affinity of the neoantigen; and can also increase with the relative HLA binding affinity (neoantigen vs. wildtype counterpart); and can increase with the number of HLA alleles bound. Surprisingly, the combination of the results of at least two of these determinations or parameters, preferably at least three, at least four, at least five or six thereof, results in a ranking of potential neoantigens in which higher ranked neoantigens peptides show a surprisingly increased potential as personalized cancer vaccines. The at least two parameters after categorization are combined, i.e. suitably summed in a weighted manner. Such a weighted approach provides the additional surprising and unexpected effect of an improved ranking with neoantigens being ranked higher that show a very improved potential of being potent cancer vaccines. It was entirely unexpected that a combination could be generalized to the suggested methods as provided herein, which are generally applicable to patients having cancer without the need for individual adaptation. This is achieved by categorization of the results of the different determinations and their combination in a weighted manner.

In a preferred embodiment of the ranking/selection method for cancer-specific neoantigen ranking/selection, the combined score for each of the plurality of the potential neoantigens is determined in a manner weighted such that for at least one classifying descriptor, the class dependent contribution to the combined score will for at least one class deviate from a linear relation with class order or will be a penalty.

Using a non-linear relation between class and contribution allows classifying the neoantigen such that an estimated uncertainty of determination can best be taken into account. For example, where a calculated binding affinity is small, rounding errors that cause the same absolute error will result in a large relative change and thus the calculated binding affinity is more affected by errors. Also, where a binding affinity is extremely low, the exact overall value will be of little importance and other factors will become more important. Therefore, it is reasonable to disregard seemingly or actual existing differences and only consider values that are sufficiently large. Accordingly, it is reasonable to choose the range such that in a low range, the contribution to an overall score is small for values within that range. It may also be possible to distinguish the weight of a low affinity that albeit near zero leads to a small but perceptible binding while values of binding affinity that are almost imperceptible and are thus easily outweighed by other factors will contribute significantly less. The number of classes may be larger than three, but using three classes already gives very good results and simplifies a variety of steps in the procedure.

In a preferred embodiment the ranking/selection method is executed as a computer-aided ranking/selection method wherein at least one of the steps of determining at least one classifying descriptor relating to the binning of a value, determining at least one value subjected to binning to obtain a classifying descriptor, determining a combined score for at least some of the neoantigens, ranking the plurality of at least four potential neoantigens based on the combined scores determined, filtering potential neoantigens, determining the indicative descriptor indicating whether the neoantigen is known to reside within a cancer-related gene or whether the neoantigens is not known to reside within a cancer-related gene, providing an individual library of potential neoantigens in particular as a result of at least one of biological sequence data, in particular at least one of DNA sequence data, RNA sequence data, protein sequence data, or peptide sequence data, in particular a combination of such data, and/or sequence data obtained from one of the subject-specific biological tumor samples or from such tumor material and additionally subject-specific biological non-tumor material, which are obtained in particular by high-throughput DNA sequencing of at least a number of genes, preferably all genes, high-throughput sequencing of messenger RNA (mRNA) molecules or total RNA, and/or by protein or peptide sequence analysis using e.g. tandem mass spectrometry (in particular by proteomics and/or ligandomics), is a step computer aided or implemented.

It should thus be noted that usually at least some, typically most and frequently all steps of the selection and/or ranking method may and shall take place in a computer aided manner. In most cases, implementing such steps in a computer aided manner is far more than a mere convenience. Obtaining results in a sufficiently fast manner usually is vital in the literal meaning of the word as calculating the results without computer support while theoretically feasible would not only be prohibitively expensive but might also lead to a patient having cancer dying before the result is obtained. This holds in particular true for in-silico determination of e.g. the allele frequency of the at least one tumor-specific mutation in the neoantigen of the subject, a relative expression rate of the at least one variant within a neoantigen in one or more cancerous cells of the subject, a binding affinity of a neoantigen to a particular HLA allele present according to the subject's HLA type, a relative HLA binding affinity of the subject specific potential neoantigen as compared to the corresponding non-mutated wild-type sequence, a binding affinity to more than one HLA allele present according to the subject's HLA type, the HLA promiscuity of a neoantigen, the reliability of predicting binding of the subject specific potential neoantigen to a HLA allele of the respective patient, the classification of each predictor, the calculation of the total score of each neoantigen and the final ranking of neoantigens in an HLA-specific or-unspecific manner.

Even where it is “only” determined whether a neoantigen is known to reside within a cancer-related gene or whether the neoantigen is not known to reside within a cancer-related gene, the determination will involve a comparison with existing database entries relating to information which genes are known to be cancer-related. It should be noted that for such a comparison, even if time needed could be disregarded, use of a computer may be considered vital as well, given that the comparison if done by a human being will be exhausting which in turn leads to errors that might turn out to be fatal even if for no reason other than the fact that a pharmaceutical composition might be produced that due to the errors is not improving the health of the patient. Thus, also in this regard computer-implementation of certain steps should be considered far more than a mere convenience.

In more detail, it is also noted that, within the present invention, it may be determined whether a given neoantigen is known to originate from a cancer-related gene or even harbors a cancer driver or drug resistance mutation. Cancer-related genes as well as cancer driver or drug resistance mutations are known to the person skilled in the art from various available data banks including, but not limited to, COSMIC (the Catalogue of Somatic Mutations in Cancer), CCGD (the Candidate Cancer Gene Database), ICGC (International Cancer Genome Consortium), TGDB (the Tumor Gene Database), PMKB (Precision Medicine Knowledgebase), OncoKB My Cancer Genome or those made available by Galperin et al. (2016) Nucleic Acid Research 45, Issue D1, pp. D1-D11.

COSMIC, the Catalogue of Somatic Mutations in Cancer, is a project of the Wellcome Sanger Institute (WSI). WSI is operated by Genome Research Limited (GRL), a charity registered in England with the number 1021457 and a company registered in England with number 2742969, whose registered office is 215 Euston Road, London, NW1 2BE.

CCGD is the Candidate Cancer Gene Database is a product of the Starr Lab at the University of Minnesota (UMN). An in-depth description of this database was published in Nucleic Acids Res. 2015 January; 43 (Database issue):D844-8. doi: 10.1093/nar/gku770. Epub 2014 Sep. 4. The Candidate Cancer Gene Database is a database of cancer driver genes from forward genetic screens in mice.

ICGC is the International Cancer Genome Consortium, a voluntary scientific organization that provides a forum for collaboration among the world's leading cancer and genomic researchers. The ICGC was launched in 2008 to coordinate large-scale cancer genome studies in tumors from 50 cancer types and/or subtypes that are of main importance across the globe. The ICGC incorporates data from The Cancer Genome Atlas (TCGA) and the Sanger Cancer Genome Project. The consortium's secretariat is at the Ontario Institute for Cancer Research in Toronto, Canada, which will also operate the data coordination center.

TGDB (the Tumor Gene Database), is provided by the Baylor College of Medicine, One Baylor Plaza, Houston, Texas. For further details relating to the PMKB (Precision Medicine Knowledgebase), reference is made to Huang et al. (2017) The cancer precision medicine knowledge base for structured clinical-grade mutations and interpretations.J Am Med Inform Assoc. 2017 May 1;24 (3):513-519. doi: 10.1093/jamia/ocw148. L,.

Onco KB is a Precision Oncology Knowledge Base containing information about the effects and treatment implications of gene alterations in 642 specific cancer genes, including such alterations which are predictive of response to approved drugs in specific cancer indications. The information is curated from various sources, such as guidelines from the FDA, NCCN, or ASCO, ClinicalTrials.gov and the scientific literature. The database is developed and maintained by the Knowledge Systems group in the Marie Josée and Henry R. Kravis Center for Molecular Oncology at Memorial Sloan Kettering Cancer Center (MSK), in partnership with Quest Diagnostics and Watson for Genomics, IBM.

Also, it should be noted that a database compilation can be established comprising information from different sources such as several of the above mentioned databases and/or results from own research. In the examples, reference will be found to such a database.

Accordingly, the skilled person is able to determine whether the sequence of a potential neoantigen is located within a known cancer-related gene or whether it contains a cancer driver or drug resistance mutation. A descriptor attributed to the respective neoantigen may change, in particular increase with the probability that a potential neoantigen is located within a known cancer-related gene or contains a cancer driver, or drug resistance mutation. In one embodiment, there need only be two discrete values attributed to parameter indicating whether the potential neoantigen originates from a known cancer-related gene or not. In another embodiment, there need only be two discrete values attributed to parameter indicating whether the potential neoantigen contains a cancer driver mutation or drug resistance mutation or not.

Even the binning and ranking itself may be bothersome if a large number of neoantigens and/or a large number of descriptors are considered. Thus, here, computer-assistance may be preferable as well.

Within the present invention, where the allele frequency of the at least one tumor-specific mutation in the neoantigen in the tumor of the subject is considered, this is based on the assumption that with high allele frequency in the tumor, the neoantigen is more likely to be present in a high proportion of the tumor cells. Accordingly, the importance and hence overall score contribution attributed to a corresponding parameter increases with the allele frequency in which the tumor-specific mutation is present in the analyzed sample. The allele frequencies of all tumor-specific variants directly depend on the tumor content of the tumor sample analyzed. For example, if only half of the cells in a tumor sample are indeed cancerous cells and the other half of the cells are normal cells, the allele frequency of tumor-specific variants usually cannot be higher than 50% (homozygous variants) or 25% (heterozygous variants) in this sample. However, in some cases copy number alterations may affect the allele frequency of tumor-specific mutations. In a preferred embodiment of the invention, the allele frequency descriptor is chosen according to threshold values determined for high, medium and/or low allele frequency. For example, a high allele frequency may correspond to a value higher or equal to ⅔ times half the tumor content, while a low allele frequency may correspond to a value lower as ⅓ times half the tumor content and values in between may correspond to a medium allele frequency.

Then, it will be noted by a person skilled in the art that filtering out potential neoantigens prior to the ranking/selection or handicapping their combined score based on a neoantigen peptide length; a value relating to the neoantigen showing identity to any self-peptide or showing no identity to any self-peptide; a value relating to the overall expression rate of the gene harboring the neoantigen; a value relating to the neoantigen hydrophobicity; and/or a value relating to the homopolymeric amino acid stretches contained within the neoantigen sequence may also require lengthy calculations and/or tedious comparison with data base entries. Therefore, here, implementation as a computer aided method step again may be considered at least helpful if not vital as well.

Furthermore, it should be noted that even a computer aided classification, binning and/or determining an overall score from a limited number of neoantigens can be considered vital as implementing these steps as computer aided steps helps to avoid clerical errors.

In a particularly preferred embodiment of the invention, the computer aided steps are executed such that intermediate results obtained can be verified prior to neoantigen ranking/selection. Such verification could be executed using an automated expert system although in general it will be preferred to have a human control of the final ranking/selection and thus also of at least some of the intermediate results. In any case the sequencing data should preferably be visually inspected at each selected variant site in order to confirm the presence and/or expression of the respective variant and to exclude any sequencing artefact.

In a preferred embodiment of the method the indicative descriptor indicating whether the neoantigen is known to reside within a cancer-related gene or whether the neoantigen is not known to reside within a cancer-related gene has a first value if the neoantigen is known to be from a cancer-related gene and has another value lower than the first if the neoantigen does not reside in a cancer-related gene. Similarly, the respective neoantigen may be attributed a value higher than the first value if it resides within a cancer-related gene and additionally is known to carry a cancer driver or cancer drug-resistance mutation.

For certain genes the scientific evidence that they are cancer-related may not be strong. Therefore, in another embodiment the indicative descriptor whether the neoantigen is known to reside within a cancer-related gene or whether the neoantigen is not known to reside within a cancer-related gene may be divided in more than two but at least three classes and neoantigens are classified according to the likelihood that they are derived from a cancer-related gene.

In other words, it is possible to take into account that a specific neoantigen has only been assumed to be cancer-related even though the assumption has not yet been fully verified with scientific methods to a generally required level of confidence. Such a neoantigen can be distinguished from a neoantigen that has clearly and with high certainty been found to be cancer-related. It can also be distinguished from a neoantigen that may have been suspected to be cancer-related in the past, but for which sound scientific analysis of a large amount of data has indicated that with a high level of confidence despite an initial assumption to the contrary, such a given other neoantigen is not cancer-related. Thus, for a given neoantigen known to be not cancer-related, the overall score can easily be handicapped by an extremely low or even negative weight or by filtering out the neoantigen entirely from a ranking/selection. Also, by assigning a low but positive non-zero weight to a neoantigen that at the time of scoring is considered to be cancer-related even though with a level of confidence still lower than usual due to ongoing scientific evaluations, current best assumptions can be taken into account without overestimating the importance of a given neoantigen. It should be noted that the weight assigned to any given neoantigen in view of its relation to cancer, the descriptor and class and/or the binning intervals may be subject to review by a medical doctor treating a patient and/or a scientific advisor at any time and that over the course of time, inevitably chosen values need be altered as scientific progress is made. Within the present invention, a difference may also be made between neoantigens that are not known to reside within a cancer-related gene and those that are known not to reside within a cancer-related gene, i.e. those for which information is available that the respective gene is not cancer-related.

It will thus be understood that the weight of other descriptors and/or the intervals used for their binning may be adapted over time as well.

In a preferred embodiment of the method a step is included of filtering out potential neoantigens prior to selection and/or ranking, or a step of handicapping the combined score of potential neoantigens prior to ranking is included, the handicapping or filtering being in particular based on a value relative to the neoantigen peptide length; a value relating to the neoantigen being a self-peptide or not being a self-peptide; a value relating to the neoantigen expression rate; a value relating to the expression rate of the gene in which the neoantigen resides; a value relating to the neoantigen hydrophobicity; a value relating to the neoantigen poly-amino acid stretches and/or values relating to specific peptide motifs determining the stability, oxidation susceptibility or manufacturability of a neoantigen.

In this respect, the average skilled person will be aware that according to a present understanding certain neoantigens should not be ranked/selected e.g. because the chemical properties thereof are considered to be highly disadvantageous for administering a treatment. In order to prevent that such neoantigens are selected, it is possible to either filter them out before scoring and/or before determining values a descriptor used in scoring is based upon. However, it may be advantageous to include such neoantigens for further considerations rather than filter them out despite certain current concerns. In such a case, the overall score of such neoantigens might be handicapped to an extent sufficient to avoid that they are selected. This may be advantageous in particular as it allows re-evaluation of the overall result should at later times the property of the neoantigen leading to a current handicapping of its score be found to be disregardable in view of further scientific progress.

According to the present understanding, in a preferred embodiment of the invention, the method further comprises a step to ensure that prior to the selection, neoantigens are excluded for which it is likely that a low ranked position will or should be obtained. If such filtering or handicapping is done according to at least one of the parameters peptide length, self-peptides, expression rate, hydrophobicity, poly-amino acid stretches and/or other peptide motifs determining stability, oxidation susceptibility and manufacturability, this takes into account that depending on the HLA type, i.e. HLA I or HLA II, to which binding of the neoantigens is restricted, peptide length is known to play an important role. Thus, neoantigens lying outside of lengths of potentially bound peptides by either HLA I or HLA Il type proteins can be excluded in a preferred manner. This helps to improve the ranking/selection. In a preferred embodiment of the invention, for HLA I restricted peptides, those are excluded that do not comprise between 8 to 11 amino acid residues. For HLA II restricted peptides, it is preferred to exclude those that do not have a length of between 12 and 32 amino acid residues. With respect to self-peptides, it is preferred to exclude those which are known to be part of an endogenously present wildtype sequences. With respect to the expression rate, it is preferred to exclude those neoantigens which are not expressed in the tumor. If neoantigens are converted to peptides for e.g. cancer vaccine production the subsequent additional filter criteria have found to be useful, in order to ensure the stability, manufacturability and solubility of such peptides. If neoantigens are delivered by other methods using e.g. viral vectors, RNA or DNA encoding neoantigens, the subsequent filter criteria may be less relevant. With respect to hydrophobicity of the neoantigen, it is preferred to exclude those with a high hydrophobicity, whereby high preferably relates to a percentage of more than about 64% hydrophobic amino acids in the potential neoantigen. With respect to poly-amino acid stretches, it is preferred to exclude those which contain three or more identical adjacent amino acid residues. With respect to stability it is preferred to exclude those neoantigens which contain cysteines and/or glutamine/glutamate at the N-terminus. With respect to oxidation susceptibility it is preferred to exclude those which contain one or more cysteines and/or methionines. With respect to manufacturability it is preferred to exclude those neoantigens containing glutamine or glutamate at the N-terminus as these can spontaneously cyclize to pyroglutamate. Furthermore, neoantigens containing di-amino acid motifs such as DG and/or DR should be excluded from a peptide vaccine cocktail as these are prone to aspartimide formation during peptide synthesis.

As can be seen above, binding affinity related values may be considered in selecting neoantigens according to the present invention. In particular, considering the binding affinity to particular HLA alleles, considering the relative HLA binding affinity of the neoantigen compared to a non-mutated wild-type sequence, and considering the binding affinity to more than one HLA allele present according to the subject's HLA type have been mentioned above. However, it will be understood that in certain tumor cells, certain HLA alleles usually present in the normal cells of a patient may not be present. It is advantageous if in such a case, HLA types not present in the tumor cells are excluded from analysis, i.e. binding affinity analysis as defined above.

Therefore, where in a preferred embodiment of the ranking/selection method for cancer-specific neoantigen selection at least one of a classifying descriptor relating to the binning of a value of a binding affinity to a particular HLA allele present according to the subject's HLA type, into one of at least three different classes ordered according to the intervals of values binned into each class; a classifying descriptor relating to the binning of a value of a relative HLA binding affinity of the subject specific potential neoantigen as compared to the corresponding non-mutated wild-type sequence into one of at least three different classes ordered according to the intervals of values binned into each class; a classifying descriptor relating to the binning of a value of a binding affinity to more than one HLA allele present according to the subject's HLA type, into one of at least three different classes ordered according to the intervals of values binned into each class; a classifying descriptor relating to the binning of a value of an HLA promiscuity of a neoantigen into one of at least three different classes ordered according to the intervals of values binned into each class; is determined, it is preferred that for determination of the values classified, HLA alleles for which a concentration in tumor cells derived from said subject having cancer lower than normal is assumed are excluded. For the purpose of the present invention, this can be assumed to be the case if the concentration is e.g. 5% lower, or is 10% lower or is 15% lower or is 20% lower or is 25% lower or is 50% lower or is 75% lower or is 100% lower.

Regarding binding affinity values, according to a preferred embodiment of the present invention, binding affinity related values of the respective neoantigen to particular HLA alleles present according to the subject's HLA type can be determined as part of input data.

It will be understood that scores/binding affinities can be determined by, for example, software tools. It is preferred to use data calculated by software tools such as NetMHC, NetMHCpan, SYFPEITHI, MixMHCpred, MHCnuggets, MHCflurry, and/or antigen.garnish software.

Note that both the NetMHC database and the NetMHCpan database is offered by Technical University of Denmark, DTU Bioinformatics, Kemitorvet, Building 208, DK-2800. SYFPEITHi is a database of MHC ligands and peptide motifs; see “Hans-Georg Rammensee, Jutta Bachmann, Niels Nikolaus Emmerich, Oskar Alexander Bachor, Stefan Stevanovic: SYFPEITHI: database for MHC ligands and peptide motifs. Immunogenetics (1999) 50:213-219”.

MixMHCpred prediction software has been developed and published by the David Gfeller's lab (Swiss Institute of Bioinformatics) under “Bassani-Sternberg M, Chong C, Guillaume P, Solleder M, Pak H, Gannon P O, Kandalaft L E, Coukos G, Gfeller D. Deciphering HLA-I motifs across HLA peptidomes improves neo-antigen predictions and identifies allostery regulating HLA specificity. PLOS Comput Biol. 2017 Aug. 23;13(8):e1005725”.

MHCnuggets has been developed by the lab of Rachel Karchin (Johns Hopkins University); see Bhattacharya et al. (2017) bioRxviv 154757.

MHCflurry was developed by the lab of Jeff Hammerbacher; see T. J. O'Donnell et al. (2018) Cell Systems 7(1); pp. 129-132.

The antigen.garnish software has been developed by Andrew J. Rech et al.; see Richman et al. (2019) Cell Systems.

However, any alternative method providing information with respect to the binding affinity of a neoantigen to a particular HLA allele may be used within the present invention. That is, the above exemplified tools may be supplemented and/or replaced with additional/alternative tools. Such tools rely on, for example as SYFPEITHI, a simple model (position specific scoring matrices) based on the observed frequency of an amino acid at a specific position in the peptide sequence to score novel peptides binding a specific HLA molecule. The training data of SYFPEITHI consist of peptides that are known to be presented on the cell surface via HLA molecules. Thus, the training data not only represent the ability of a peptide to bind to a specific MHC allele but also to be produced by the antigen processing pathway (proteasomal cleavage and TAP transport). NetMHC is a neural network-based machine-learning algorithm to predict the binding affinity of peptides to a specific MHC class I allele. The training data consist of experimentally determined binding affinities of peptide:MHC complexes and the sequence of know MHC ligands. NetMHC uses a complex representation of the peptides, based on sequence properties as well as physic-chemical properties of the amino acids. NetMHC can generalize MHC binding of peptides of length 8-11 from training data mostly consisting of peptides of 9 amino acids length. Thereby it increases the MHC coverage for prediction of peptides of length 9-11 (for many alleles the training data is limited to peptides of length 9). NetMHCpan is a further development of NetMHC. MHC alleles and different peptide lengths are not equally represented in the available training data. NetMHCpan leverages information across MHC binding specificities and peptide lengths and can therefore generate predictions of the affinity of any peptide-MHC class I interaction. Binding prediction is thus available for every known MHC class I allele, and not only for those sufficiently represented in the training data. The above tools are preferably used, however, the skilled person is in a position to adapt these tools to specific needs of the methods provided herein, if required. For example, as an alternative and/or in addition, it would also be possible to determine peptide-HLA I interactions, by e.g. ligandomics (elution of HLA I bound peptides and MS identification) or in vitro binding assays with peptides and HLA I molecules.

Subsequent to determining binding affinities preferably using software tools, in particular one, two, three or more of the software tools identified above, the resulting scores of the preferably more than one used software tools may be combined in order to provide a ranking of neoantigens. Obtaining a ranking based on values derived with different tools and/or models reduces errors induced by inter alia the specific model a tool implements. In the invention, this is advantageous as it contributes to obtain a ranking/selection even less influenced by errors in initial measurements or imprecise scientific assumptions and estimates.

In a preferred embodiment, threshold values are predetermined in order to provide distinct classes of affinity scores such as high, medium and low affinities for which discrete numerical values are provided.

Within the present invention, a descriptor based on the relative HLA binding affinity of the respective neoantigen as compared to the non-mutated version thereof may be considered. For that purpose, it is preferred to use the same technique as described above. In a preferred embodiment, there are discrete numerical values attributed to neoantigens for which the result lies within predetermined threshold values. For example, a relative binding affinity of the mutated neoantigen as compared with the wildtype version thereof of more than 1.1 may be attributed to a high numerical value (or large contribution to the overall score) whereas a relative binding affinity of below 0.9 may be attributed to a low numerical value (or low contribution to the overall score). In a further embodiment, for a relative binding affinity of the mutated neoantigen as compared with the wildtype version thereof a ratio of more than 2- or 3-fold higher for the neoantigen may be attributed to a high numerical value (or large contribution to the overall score) whereas a ratio below ½ or ⅓, respectively, may be attributed to a low numerical value (or low contribution to the overall score).

Within the present invention, a descriptor may be based on the number of HLA types for which binding is predicted, i.e. whether binding affinity is predicted for more than one HLA allele whereby the numerical value increases with the number of HLA types bound.

As indicated above, certain HLA alleles should be disregarded in view of a concentration thereof in a tumor cell being lower than normal. In this context, in a preferred embodiment of the ranking/selection method for cancer-specific neoantigen selection, HLA alleles are considered to be subject to an expression reduction, mutation or deletion/loss derived in view of a tumor transcriptome, a tumor exome or a tumor proteome or an immuno-histochemistry staining of a tumor tissue sample or a normal exome (e.g. from blood), a normal transcriptome, or a normal proteome, or an immunohistochemistry staining of a normal tissue sample. Thus, genetic and other data can be used to conclude that a HLA reduction or loss must be taken into account.

The methods of the present invention may comprise, as a first step, accessing or providing a library of potential neoantigens of a subject having cancer, wherein the neoantigens carry at least one tumor-specific mutation. Thus, as input data, the methods of the present invention may use exome and/or transcriptome sequencing results of the patient having cancer. These sequencing data sets preferably comprise information about somatic missense variants, i.e. non-synonymous single nucleotide variants (SNVs), non-synonymous multi-nucleotide variants (MNVs), frame shift variants (e.g. from Indels), and/or fusion genes (e.g. from chromosome translocations), the corresponding transcriptome data and the patient's HLA type. Based on this information, the methods of the present invention are able to provide a ranking of all potential neoantigens comprised as sequence information in the data sets. The skilled person is well-aware of methods suitable to obtain these data sets from the patient having cancer including sequence information received from tumor cells and healthy cells as a reference. It is preferred to use whole exome sequence data generated by methods well-known in the art (i.e. next-generation sequencing).

Once the ranking is done, a selection may take place. In this context, the average skilled person will be aware that it is possible to select more than one neoantigen. In this respect, the selection may comprise one neoantigen or more than one, for example two, three, four, five, six, seven, eight, nine, ten or more neoantigens according to their ranked position.

It is useful and preferred to select more than one neoantigen. In case more than one neoantigen is selected, care can be taken to increase the likelihood that the selection is effective by requesting that the neoantigens selected together have certain properties as an ensemble. For example, care can be taken that different HLA types are considered. Even though this may lead to a situation where an ensemble of e.g. six neoantigens is selected that do not constitute the six best scored neoantigens initially considered, the overall selection will still give better results in treating a patient because the likelihood is reduced that all neoantigens will turn out to be ineffective for unknown, unpredicted, underestimated reasons or in the case the expression or integrity of one or more of the patient's HLA alleles is reduced or lost during time or therapy. Indeed the possibility exists that an HLA allele is lost or mutated in the course of a treatment due to, e.g., immunogenic pressure. For this reason, for therapeutic purpose (e.g. for the design of a cancer vaccine) it is useful to target further neoantigens which bind to different HLA alleles. Here, targeting a set of neoantigens binding to all available HLA alleles avoids competition for binding to one certain HLA allele and immunodominance effects of one peptide over the others.

In a preferred embodiment of the selection method for cancer-specific neoantigens, the preferred method allows selecting for each HLA class I molecule of the patient at least one neoantigen and additionally HLA class Il restricted neoantigens.

Such a selection is considered to be advantageous as selecting neoantigens in view of different HLA classes is believed to increase the likelihood that a given selection is effective for treating a patient. HLA class I restricted neoepitopes more effectively lead to the activation of cytotoxic T-cells (CD8+ T cells) while HLA class Il restricted neoepitopes more effectively lead to the activation of T-helper cells (CD4+ T cells). As both T cell population have different but complementing anti-tumor actions, inducing both a CD8+ and a CD4+ T cell response is meant to be most beneficial for anti-cancer immunotherapies.

In a preferred embodiment of the ranking/selection method for cancer-specific neoantigen selection at least one classifying descriptor is binning the respective value into one of not more than five ordered classes, in particular into not more than four ordered classes, in particular preferably into one of three ordered classes.

Using a large number of ranges that a respective value can be binned into despite being seemingly more precise may not be the most preferred embodiment. On the one hand, the average skilled person will be aware given the present disclosure that a large number of influences need to be factored in. Then, a ranking initially obtained based on an overall score will not determine with absolute certainty that a given neoantigen is selected for a cocktail based on a plurality of cocktails. Accordingly, it may be advantageous to include a given neoantigen in a multi-neoantigen selection only if several factors, e.g. from other descriptors, are also met.

Therefore, although surprising, it has been found sufficient to only distinguish a small number of different ranges. Using a small number of different ranges for any given descriptor not only helps eliminate pseudo-scientific reasoning to rationalize specific thresholds and limits actually set according to personal preferences, but also allows for lower precision of in-silico evaluation of data frequently allowing fewer iterations, calculations with less precision etc. without significantly affecting efficacy of treatment with the respective neoantigen selection. This also helps to reduce the cost and time requirements of the selection method where otherwise particularly lengthy and thus expensive computations may be necessary. Therefore, a number of ranges of less than or equal to five is highly preferred. This is even the case where significantly more than four potential neoantigens are ranked, e.g. at least 5, at least 10, at least 15 or at least 20, or at least 30 potential neoantigens are ranked or at least provided from the library prior to filtering. It will be understood that even four ranges usually will suffice, allowing to distinguish a value not discriminable against a zero value, a value not discriminable against a maximum value and two intermediate values. However, in a typical example, it is sufficient and even preferred to have but one intermediate range so that only three ranges “high-medium-low” are needed.

In a preferred embodiment of the ranking/selection method for cancer-specific neoantigens, all classifying descriptors are binning the respective value into one of not more than five classes, in particular into not more than four classes, in particular preferably into one of three classes. While it is possible to have a different number of possible ranges each descriptor is binned into, a more straightforward and thus faster and cheaper approach is to use the same number of ranges for all classifying descriptors.

It has been found that the number of ranges can be reduced in particular where a sufficiently large number of different descriptors are considered, such as 4, 5, 6 or more descriptors that are all evaluated together. In such a case, there usually will exist more than one pair of descriptors a/b for which the contribution to a combined score S that is determined additively in a manner S=S(a)+S(b) is such that for at least one pair of ranges (a1,a2) of the three, four or more ranges the first descriptor may take and one pair of ranges (b1,b2) the second descriptor may take the contribution S=S(a)+S(b) to the combined score is such that S(a1)+S(b1)>S(a2)+S(b1), S(a2)+S(b1)>S(a2)+S(b2) while S(a1)+S(b2)>S(a2)+S(b1). In other words, a relation may exist such as [S(a1)+S(b1)]>[S(a1)+S(b2)]>[S(a2)+S(b1)]>[S(a2)+S(b2)]. Such property of the influence of descriptors allow to disregard minute differences between certain values as insignificant while still obtaining a very good selection.

In a preferred embodiment of the ranking/selection method for cancer-specific neoantigens the individual library of potential neoantigens is provided in response to exome and/or transcriptome sequencing of subject-specific biological material and/or by somatic missense variant identification, in particular of a fresh frozen tumor sample, formalin-fixed paraffin-embedded tumor material, a stabilized tumor sample, a tumor sample stabilized in PaxGene or Streck Tubes, circulating tumor DNA (ctDNA), or circulating/disseminated tumor cells. PaxGene is a trademark by PreAnalytix, a joint venture between Becton, Dickinson and Qiagen, located at Feldbachstrasse, CH 8634 Hombrechtikon. StreckTubes are available from Streck, 7002 S- 109^{th} Street, La Vista, Ne, 68128, United States.

As will be understood by the average skilled person, it is only necessary to provide a sequencing of certain material to obtain data the method can be based upon. It should also be noted that some of the sequencing data can be obtained using material from a patient that may not only be easily obtained but will also be sufficiently stable so as to be transported to a laboratory for sequencing or analysis.

It should be noted and will be understood that it is not necessary to obtain samples, analyze samples, analyze the data obtained by sample analysis, selecting neoantigens and using the selected antigens in preparing a pharmaceutical composition at one and the same exact location.

Where a plurality of descriptors are evaluated according to the invention, and each may contribute differently according to the respective value the descriptor has for a given neoantigen, the weight assigned to determine the ranking will preferably be such that neoantigens are not simply grouped such that all neoantigens having a first descriptor with a high value are all in one group, all neoantigens having an intermediate value are in a lower ranked group and all neoantigens having a low value are in a third group, and then in each of these groups a second descriptor exists that again splits each (sub) group according to the value this descriptor has etc. until all descriptors are considered. Rather, there usually and preferably will be a situation where the weights each descriptor is assigned in a value-dependent matter is such that a mixing occurs depending on the exact value and the weight assigned. In mathematical terms, thus for at least two descriptors a/b contributing to a combined score S additively in a manner S=S(a)+S(b), at least one pair of values (a1,a2) the first descriptor may take and one pair of values (b1,b2) the second descriptor may take exists such that the contribution S(a)+S(b) to the combined score is such that S(a1)+S(b1)>S(a2)+S(b1), S(a2)+S(b1)>S(a2)+S(b2) while S(a1)+S(b2)>S(a2)+S(b1). In other words, a relation may exist such as [S(a1)+S(b1)]>[S(a1)+S(b2)]>[S(a2)+S(b1)]>[S(a2)+S(b2)].

It is noted that usually a plurality of pairs of descriptors exist that have such a property, in particular at least 2, 3 or 4 pairs of descriptors and that in a particularly preferred embodiment for at least one descriptor at least two such pairs can be found.

In a preferred embodiment of the ranking/selection method for cancer-specific neoantigens, this may be achieved inter alia if the maximum possible contribution to the combined score of the descriptor relating to indicating whether or not the neoantigen is known to be cancer-related is larger than the maximum possible contribution to the combined score of any single of the descriptors relating to a relative expression rate in one or more cancerous cells of the subject, a binding affinity to a particular HLA allele present according to the subject's HLA type, a relative HLA binding affinity of the subject specific potential neoantigen as compared to the corresponding non-mutated wild-type sequence, a binding affinity to more than one HLA allele present according to the subject's HLA type, an HLA promiscuity and the reliability of predicting binding of the subject specific potential neoantigen; and/or wherein the maximum possible contribution to the combined score of the descriptor relating to a relative expression rate in one or more cancerous cells of the subject is larger than the maximum possible contribution to the combined score of any single of the descriptors relating to a binding affinity to particular HLA alleles present according to the subject's HLA type, a relative HLA binding affinity of the subject specific potential neoantigen as compared to the corresponding non-mutated wild-type sequence, a binding affinity to more than one HLA allele present according to the subject's HLA type, an HLA promiscuity, and the reliability of predicting binding of the subject specific potential neoantigen; and/or wherein the maximum possible contribution to the combined score of the descriptor relating to a binding affinity to a particular HLA allele present according to the subject's HLA type is larger than the maximum possible contribution to the combined score of any single of the descriptors relating to a relative HLA binding affinity of the subject specific potential neoantigen as compared to the corresponding non-mutated wild-type sequence, a binding affinity to more than one HLA allele present according to the subject's HLA type, an HLA promiscuity, and the reliability of predicting binding of the subject specific potential neoantigen; and/or wherein the maximum possible contribution to the combined score of the descriptor relating to a relative HLA binding affinity of the subject specific potential neoantigen as compared to the corresponding non-mutated wild-type sequence is larger than the maximum possible contribution to the combined score of any single of the descriptors relating to a binding affinity to more than one HLA allele present according to the subject's HLA type, an HLA promiscuity, and the reliability of predicting binding of the subject specific potential neoantigen; and/or wherein the maximum possible contribution to the combined score of the descriptor relating to a binding affinity to more than one HLA allele present according to the subject's HLA type is larger than the maximum possible contribution to the combined score of any single of the descriptors relating to an HLA promiscuity and the reliability of predicting binding of the subject specific potential neoantigen; and/or the maximum possible contribution to the combined score of the descriptor relating to an HLA promiscuity is larger than the maximum possible contribution to the combined score of the descriptors relating to the reliability of predicting binding of the subject specific potential neoantigen. Regarding the reliability of predicting binding, it should be noted that usually binding affinities are numerically calculated using a model and that different models could be used in calculating binding affinities. If more than one model or method of calculation is used, it is likely that the binding affinities calculated with one model will deviate somewhat from binding affinities calculated with another model. Such deviations can be evaluated to determine a reliability of predicting binding, e.g. by considering the absolute or relative difference, the mean variation where a larger number of models are used, and so forth.

It should be noted that in a preferred embodiment of the ranking/selection method for cancer-specific neoantigen selection an ensemble consisting of a plurality of different neoantigens is selected. In such a case, the neoantigens of the ensemble can be selected in view of their ranking such that for each of a plurality of the HLA alleles considered the (nonfiltered or filtered) most favorably ranked neoantigen is selected, preferably for each HLA allele the (nonfiltered or filtered) most favorably ranked neoantigen is selected, and such that, if the ensemble comprises more neoantigens than these most favorably ranked neoantigens, then further neoantigens for different alleles are selected starting with HLA-A or B alleles;
and preferably further such that if at least two such neoantigens for the same variant, but different alleles starting with HLA-A or B alleles are equally ranked, then a neoantigen thereof with an HLA allele hitherto underrepresented in the ensemble is selected, and preferably further such that if at least two such neoantigens exist binding to no hitherto underrepresented HLA allele, then a neoantigen thereof with a higher HLA binding affinity is selected, preferably a higher binding affinity not according to the classifying descriptor but according to the original value classified; and preferably further such that if at least two such neoantigens having an equal HLA binding affinity exist, then the neoantigen thereof having a higher HLA promiscuity is selected and preferably further such that if at least two such neoantigens having an equal HLA promiscuity exist, then the neoantigen thereof having a lower hydrophobicity is selected;
and preferably further such that if at least two such highly ranked neoantigens for different variants, but the same HLA allele are equally ranked, then the neoantigen thereof having the higher expression is selected; and preferably further such that if at least two such neoantigens having an equal expression exist, then the neoantigen thereof with a higher HLA binding affinity is selected, preferably a higher binding affinity according to not the classifying descriptor, but according to the original value classified; and preferably further such that if at least two such neoantigens having an equal HLA binding affinity exist, then the neoantigen thereof having a higher HLA promiscuity is selected and preferably further such that if at least two such neoantigens having an equal HLA promiscuity exist, then the neoantigen thereof having a lower hydrophobicity is selected.

Thus, it will be noted that there is no guarantee that a neoantigen scoring rather high actually is selected into an ensemble. Rather, the actual selection may depend on properties other high scoring neoantigens have. However, it will be understood that the final process of selecting neoantigens for an ensemble also can be computer implemented and hence automated in particular in view of the additional conditions defined above.

In a preferred embodiment of the ranking/selection method for cancer-specific neoantigen selection at least 3 neoantigens are selected. It should be noted that selecting more than one neoantigen is helpful as despite a favorable ranking a situation may occur where other unfavorable factors are not considered at all resulting in a ranking where the highest ranked neoantigen are burdened by such unfavorable factors not considered. The risk of selecting several neoantigens that all are high-ranked but burdened by unfavorable factors however is extremely low. Therefore, selecting at least three neoantigens is preferred and a larger number is even preferred. However, cost may become prohibitive if too large a number of neoantigens is selected. The best number of neoantigens selected may thus not only depend on the specific patient, the progress of his disease and thus the necessity to improve his health faster, but also on the cost of using a large plurality of neoantigens in a pharmaceutical composition rather than using a smaller plurality. The most suitable number of neoantigens selected may also depend on the delivery mechanism. Viral vectors, DNA or RNA may allow to encode and deliver high numbers of neoantigens, while vaccines consisting of individual neoantigen-resembling peptides may be restricted to up to 20 or 30 peptides per patients, due to costs, timely manufacturability and practical reasons like vaccine QC and delivery in several sub-ensembles.

Regarding different contributions of different ranges of different classifying descriptors, it has been found to be preferred for the selection method for cancer-specific neoantigen selection that a classifying descriptor relating to the binning of a value indicative for the allele frequency of the at least one tumor-specific mutation in the neoantigen of the subject into one of at least three different classes ordered according to the intervals of values binned into each class is determined such that a tumor content Y is defined and the value of the allele frequency is defined to be in the highest class if the allele frequency is at least ⅓ of the half tumor content, to be in the lowest class if the allele frequency is no more than ⅙ of half the tumor content Y and else to be in the medium class, and the maximum contribution of the corresponding classifying descriptor if the allele frequency is in the medium class being less than the contribution in case of a highest class and more than the contribution in case of a lowest class. It is noted that while “⅓” and “⅙” are useful limits for the ranges, deviations are possible, e.g. by about 5% or 10% or 15% or 25% of the values indicated. It should be noted that here, reference may be made to either half the tumor content if the somatic mutations in tumor cells are heterozygous or the total tumor content if the somatic mutations are homozygous.

It should be noted that it is possible to re-use respective data and/or intermediate data relating to selection results repeatedly. In particular, it is possible to either re-use the overall selection result repeatedly, for example because a personalized medical treatment is to be effected repeatedly based on the same given selection and/or because the selection result are to be stored together with other patient data as part of a data base that in the end can be used to improve the treatment of the patient or of other patients having a similar diagnosis. It will be understood that a data carrier comprising such a data base will have a significant economical value reflecting the wealth of scientific data included therein and that allowing access to a data base may constitute a source of significant financial income. Access may be provided in an anonymized manner. Providing data in a manner allowing their entry into such a data base is thus considered to be a significant step of both the method of the invention and the production of a data carrier including data relating to a data base that is combining anonymized or non-anonymized patient data and selection related data, in particular binnable values of descriptors usable in the method of selection. Thus, data relating to a selection method for cancer-specific neoantigen selection may be considered a vital and essential part to carry out the method and a vital means to execute the method. It is also possible to store not just the ranking and/or the selected neoantigens but to store intermediate results instead or in addition to the selection. By storing intermediate results such as the values of the descriptors, it becomes possible inter alia to re-classify descriptors to other bins, to change the weight assigned to specific descriptors or to change the number of selected neoantigens. All these measures may help to improve personalized selection methods in the future as scientific progress is made. Therefore, use of the data extends beyond one-time use.

Furthermore, it is obvious that any data obtained is intended to be used to create new products such as personalized pharmaceuticals and/or man-and/or machine-readable prescriptions for such pharmaceuticals. It is envisioned that prescriptions based on the selection may be automatically producible using such data.

It should also be noted that data obtained e.g. by in-silico analysis of genetic data as a step in neoantigen ranking/and or selection of the present invention can be made perceptible by a range of different methods, such as by visualization of data base entries on a monitor or by printing out the results or intermediate. In particular, the limited number of different ranges each descriptor is binned into allows to generate a display where the different range values or score contributions are indicated by different colors. For example, where three different ranges such as high-medium-low are used to bin the value a descriptor may have, it would be possible to assign the colors green, yellow, or red. Then, for a number of neoantigens or for all neoantigens, the weight of a particular descriptor could be used to determine a size of a specifically colored area. For example, where a value of a descriptor is binned into a high range indicating that the neoantigen might be selected in view of this descriptor, the area could be green and if at the same time the descriptor is particularly important such as if the neoantigen is known to be cancer-related, then the green area shown could be made correspondingly large. In this way, a display could be generated where for the respective neoantigens the overall red, yellow and green areas could be shown such that a large green area shows that overall the respective neoantigen should be favored whereas a large red area shows that the respective neoantigen should be disfavored.

It will be obvious that other ways of visualization exist. For example, other colors could be used, the intensity rather than the size of an area could be used to indicate whether or not a neoantigen should be selected, the areas for each descriptor could be shown spaced apart rather than in contact with each other and so forth. However, it will be obvious to the average skilled person that the specific way the computer-implemented method of the invention suggests allows to visualize the intermediate results in a way particularly easy to control. This is an advantage of the present invention as control of intermediate results will not only simplify the implementation of the computer-aided method but will also improve the confidence a user and/or a patient has in the method thus increasing acceptance.

Given the above, protection is also sought for a pharmaceutical composition comprising at least one substance determined in response to a result of a selection method as described and disclosed herein. The pharmaceutical composition of the invention may, in one embodiment, be used for treating cancer. In a further embodiment of the invention, the pharmaceutical composition of the invention may be combined with other treatments such as radiation therapy and/or with one or more further pharmaceuticals such as chemotherapy and/or anti-angiogenic drugs (e.g. Axitinib (Inlyta), Bevacizumab (Avastin), Cabozantinib (Cometriq), Everolimus (Afinitor), Lenalidomide (Revlimid), Lenvatinib mesylate (Lenvima), Pazopanib (Votrient), Ramucirumab (Cyramza), Regorafenib (Stivarga), Sorafenib (Nexavar), Sunitinib (Sutent), Thalidomide (Synovir, Thalomid), Vandetanib (Caprelsa) and/or Ziv-aflibercept (Zaltrap)) and/or targeted therapies (like Afatinib (Gilotrif), Brigatinib (Alunbrig), Cetuximab (Erbitux), Cobimetinib (Cotellic), Dabrafenib (Tafinlar), Everolimus (Afinitor), Imatinib (Gleevec), Lapatinib (Tykerb), Olaparib (Lynparza), Osimertinib (Tagrisso), Palbociclib (Ibrance), Regorafenib (Stivarga), Rituximab (Rituxan, Mabthera), Rucaparib (Rubraca), Trametinib (Mekinist), Trastuzumab (Herceptin), Vemurafenib (Zelboraf) and/or immunotherapies like immune checkpoint inhibitors (e.g. targeting CTLA-4, PD-1, PD-L1 and/or targeting other immune checkpoints like CD27, CD28, CD40, CD137, GITR, ICOS, OX40, (all stimulatory immune checkpoints), A2AR, CD272, CD276, IDO, KIR, VTCN1, LAG3, TIM-3, NOX2, VISTA (all inhibitory immune checkpoints)) and/or oncolytic viruses (like talimogene laherparepvec (T-VEC, Imlygic), pelareorep (Reolysin), HF10 (Canerpaturev—C-REV) and CVA21 (CAVATAK))-. Preferably the pharmaceutical composition of the invention may be combined with immune checkpoint inhibitors like pembrolizumab (Keytruda), nivolumab (Opdivo), cemiplimab (LIBTAYO), ipilimumab (Yervoy), atezolizumab (Tecentriq), avelumab (Bavencio), durvalumab (Imfinzi), Tremelimumab and/or Spartalizumab. The skilled person is well-aware of formulations for pharmaceutical compositions and ways how to optimize formulations for therapeutic use. Furthermore, the skilled person is well aware how such pharmaceutical compositions may be administered and how to optimize administration routes for the best therapeutic result. For example, the pharmaceutical composition of the invention may be administered intradermally, subcutaneously, intra-muscularly, intra-venously or near to or into lymphoid organs like thymus, bone marrow, spleen, tonsils or lymph nodes. It may be preferable to administer the pharmaceutical composition at a site close to the tumor or close to or into the tumor draining lymph node in order to increase the local concentration at the tumor site. The skilled person is also aware of suitable treatment regimens. In this respect, it is preferred that the pharmaceutical composition of the invention is administered continuously, e.g. every four weeks after an initial starting phase with more frequent administration. The skilled person will also be aware of the advantages to be gained by administering on ore more adjuvants before, after or together with, the pharmaceutical composition or as part of the pharmaceutical composition.

Furthermore, protection is also sought for using one or more neoantigens selected in accordance with a method as described and disclosed herein in preparing a personalized pharmaceutical composition.

Then, protection is also sought for a data carrier comprising data relatable to at least one individual patient having cancer, the data carrier carrying data relating to a plurality of potential neoantigens carrying at least one mutation considered to be specific to the cancer of the at least one individual patient in that for each of at least four potential antigens of this plurality of neoantigens at least two of the group (a) thru (h) are provided, with the group (a) thru (h) consisting of (a) an indicative descriptor indicating whether the neoantigen is known to reside within a cancer-related gene or whether the neoantigen is not known to reside within a cancer-related gene and/or a value indicative for a likelihood estimate the neoantigen has to be or has not to be cancer-related; (b) a classifying descriptor relating to the binning of a value indicative for the allele frequency of the at least one tumor-specific mutation in the neoantigen of the subject into one of at least two different classes ordered according to the intervals of values binned into each class and/or a value indicative for the allele frequency of the at least one tumor-specific mutation in the neoantigen of the subject into one of at least three different classes, ordered according to the intervals of values binned into each class; (c) a classifying descriptor relating to the binning of a value indicative for a relative expression rate of the at least one variant within a neoantigen in one or more cancerous cells of the subject into one of at least two, preferably at least three different classes ordered according to the intervals of values binned into each class and/or a value indicative for a relative expression rate of the at least one variant within a neoantigen in one or more cancerous cells of the subject; (d) a classifying descriptor relating to the binning of a value indicative for a binding affinity of a neoantigen to a particular HLA allele present according to the subject's HLA type, into one of at least three different classes, ordered according to the intervals of values binned into each class and/or a value indicative for a binding affinity of a neoantigen to a particular HLA allele present according to the subject's HLA type; (e) a classifying descriptor relating to the binning of a value indicative for a relative HLA binding affinity of the subject specific potential neoantigen as compared to the corresponding non-mutated wild-type sequence into one of at least three different classes ordered according to the intervals of values binned into each class and/or a value indicative for a relative HLA binding affinity of the subject specific potential neoantigen as compared to the corresponding non-mutated wild-type sequence; (f) a classifying descriptor relating to the binning of a value indicative for a binding affinity to more than one HLA allele present according to the subject's HLA type, into one of at least three different classes, ordered according to the intervals of values binned into each class and/or a value indicative for a binding affinity to more than one HLA allele present according to the subject's HLA type; (g) a classifying descriptor relating to the binning of a value indicative for the HLA promiscuity of a neoantigen into one of at least three different classes, preferably at least three different classes, ordered according to the intervals of values binned into each class and/or a value indicative for the HLA promiscuity of a neoantigen; (h) a classifying descriptor relating to the binning of a value indicative for the reliability of predicting binding of the subject specific potential neoantigen to a HLA allele of the respective patient into one of at least three-different classes, preferably at least three different classes, ordered according to the intervals of values binned into each class and/or a value indicative for the reliability of predicting binding of the subject specific potential neoantigen to a HLA allele of the respective patient; and/or the data carrier carrying data relating to neoantigen scoring as obtained by one of the previously claimed methods; and/or the data carrier carrying data relating one or more neoantigens selected according to one of the preceding claims; and/or the data carrier carrying data relating to instructions to produce a pharmaceutical composition comprising at least one substance determined in response to a result of a selection method as described and disclosed herein. The data carrier may comprise an entire data base or part thereof.

Furthermore, protection is sought for a kit comprising at least one of a container for biological material prepared in a manner allowing determination of personalized data usable as input into a ranking and/or selection method as disclosed herein and obtained from a patient having cancer or a data carrier storing personalized (genetic) data usable as individual-related input into a ranking and/or selection method as disclosed herein; the kit also comprising an information carrier carrying information relating to the identification of the patient, the kit further comprising instructions to execute a method according to one of the preceding method claims and/or to provide data for the production of a data carrier as described and disclosed herein.

The invention and the method of selecting neoantigens will now be disclosed in more detail.

### Definitions

Unless otherwise defined, understandable and/or obvious from the above, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The term “preferably” is used to describe features or embodiments which are not required in the present invention but may lead to improved technical effects and are thus desirable but not essential.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), Current Protocols in Immunology and Current Protocols in Human Genetics, Wiley press, and/or Harlow and Lane Antibodies: A Laboratory Manual, Cold Spring Harbor Laboratory Press, Cold Spring Harbor, N.Y. (1990).

While aspects of the invention are illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below. The invention also covers all further features shown in the figures individually, although they may not have been described in the previous or following description. Also, single alternatives of the embodiments described in the figures and the description and single alternatives of features thereof can be disclaimed from the subject matter of the other aspect of the invention.

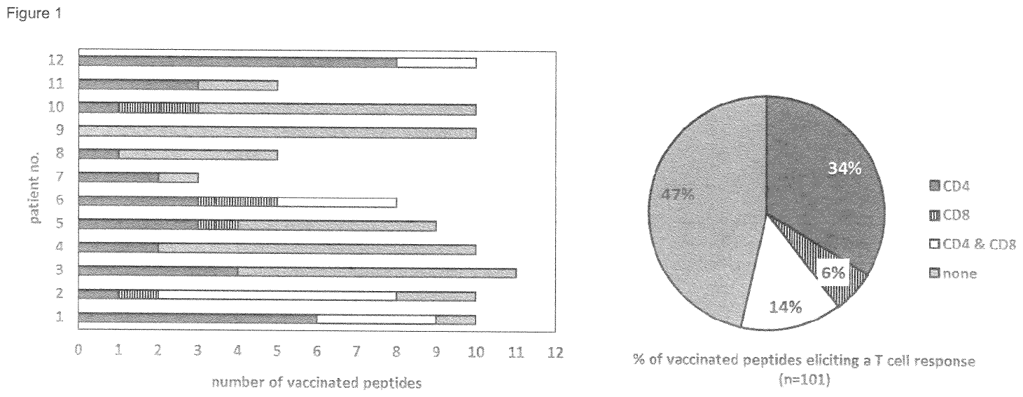

FIG. 1: Immune responses towards vaccinated neoantigen-resembling peptides (n=101) in 12 cancer patients.

Cancer patients were vaccinated for at least 2 months with neoantigen-resembling peptides selected according to the described methods. The immunostimulatory adjuvant GM-CSF was co-applied. PBMCs were isolated in the course of the vaccination. Neoantigen vaccine-specific T cell responses were detected after 11 days in vitro stimulation of the patients PBMCs (peripheral blood mononuclear cells) with single neoantigen-resembling peptides, followed by a short incubation with the same peptides or DMSO (Control) and intracellular cytokine staining and FACS analysis to quantify the T cell activation markers IFN-g, TNF, CD154 and CD107a or IL-2 in CD4+ and CD8+ T-cells.

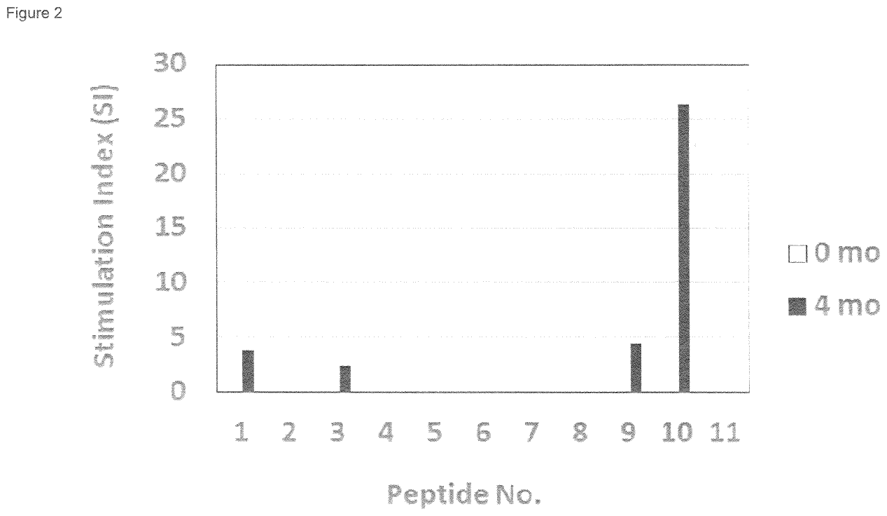

FIG. 2: Increase of neoantigen-specific T cell responses in the course of vaccination. Immune responses to vaccinated neoantigen-resembling peptides were measured as described in FIG. 1 before (0 months) and after 4 months of vaccination.

Peptide-specific responses were further evaluated using the stimulation index (SI). The stimulation index is the calculated ratio of polyfunctional activated CD4+ or CD8+ T cells (positive for at least 2 markers of CD154, IFN-y, TNF, and/or IL-2) in the peptide-stimulated sample to the negative control sample (DMSO). The graph shows that immune responses increased in the course of vaccination (data of one exemplary cancer patient are shown).

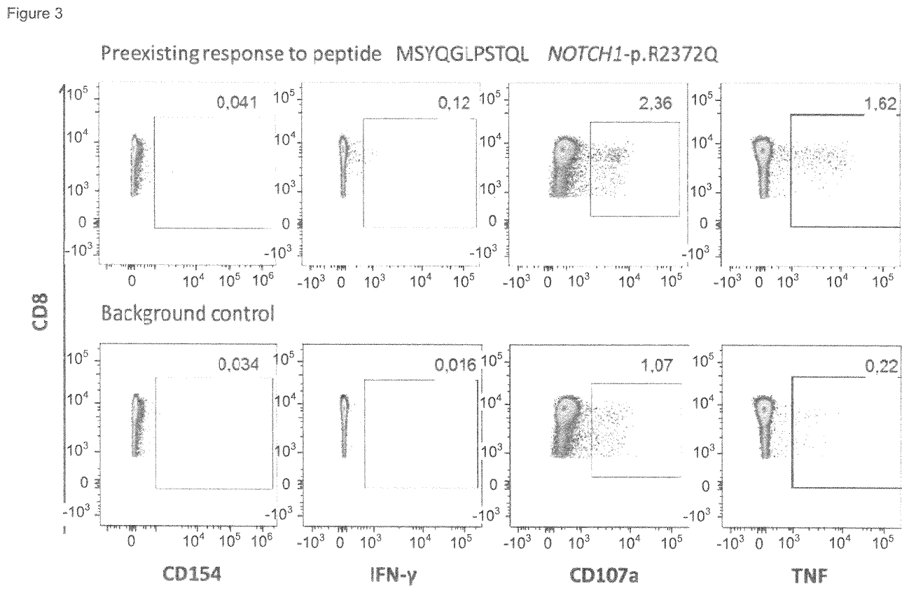

FIG. 3: Detection of preexisting T cell responses against neoantigens selected by the described methods.

For patient No. 2, preexisting CD8⁺ and CD4⁺ T cell responses were detected against 3 and 2 neoantigen-resembling peptides, respectively. Here results for one exemplary peptide are shown which were obtained before vaccination started.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

The following are examples of methods and compositions of the invention. It is understood that various other embodiments may be practiced, given the general description provided above.

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those skilled in the art that the techniques disclosed in the examples which follow represent techniques used in the present invention to function well in the practice of the invention, and thus can be considered to constitute preferred modes for its practice. However, those skilled in the art should appreciate, in light of the present disclosure that many changes can be made in the specific embodiments which are disclosed and still obtain a like or similar result without departing from the spirit and scope of the invention. A number of documents including patent applications, manufacturer's manuals and scientific publications are cited herein. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### Example 1—General Method Outline

Step 1: Determination of tumor-specific (passenger & driver) mutations by comparison of exome sequencing data from tumor and normal tissue:
   Non-synonymous Single Nucleotide Variants (SNV) and Multiple Nucleotide Variants (MNVs) in close proximity
   Indels (leading either to MNVs or to frame shifts giving rise to completely novel amino acid sequences)
   Fusion genes potentially leading to novel antigens at the breakpoint
Step 2: Definition of all possible mutated peptides which can be derived from the tumor-specific mutations found in step 1 and their genomic sequence context. For the design of such mutated peptides for each tumor-specific non-synonymous variant other non-synonymous tumor-specific or germline variants deviating from the human reference genome, which are located in the near neighborhood and on the same chromosome as the respective variant, are preferably taken into account.
Step 3: Determination of patient's HLA class I and/or class II type
   For example, based on the exome data of normal tissue.
Step 4: Identification of mutated peptides that are likely to be presented on the surface of tumor cells (neoantigens) based on the list of mutated peptides from step 2 and the HLA status from step 3.
This can be done for short peptides based on HLA class I type and/or for long peptides based on HLA class Il type of the respective patient.
For example, neoantigenic HLA class I restricted epitopes with a length of 8-11 amino acids can be predicted using the methods SYFPEITHI, NetMHC, and NetMHCpan.
For prediction of long neoantigenic epitopes (12-32 amino acids) potentially binding to HLA class II molecules algorithms like NetMHCII and NetMHCIlpan may be applied. As such algorithms are at present less reliable than those predicting short class I restricted epitopes, class II restricted epitopes can also be designed manually: from non-synonymous tumor-specific SNVs peptides of e.g. 17 amino acids can be derived in which the altered amino acid residue resides in the center position and is flanked by 8 amino acids to either side. If variants leading to frameshifts are addressed such peptides need to either cover the breakpoints (wt/mutant sequence) or any sequence downstream of the frameshift mutation but upstream of the next stop codon of the new frame. If variants leading to fusion genes are addressed such peptides need cover the breakpoints (DNA locus 1/DNA locus 2).
Step 5: Potential neoantigens homologous to any human wild-type protein listed in the UniProtKB/Swiss-Prot Database are excluded.
Step 6: Exclusion of mutated peptides which are unlikely to be expressed in the particular tumor entity or the patient's individual tumor. This can, for example, be based on:
   Tumor specific gene expression databases (e.g. The Human Protein Atlas)
   Transcriptome analysis allows to control the expression/presence of the variant in the tumor
   If possible ligandome analysis may proof the existence of respective mutated peptides on the cancer cell surface (i.e. by peptide/HLA-immunoprecipitation, peptide elution and identification by mass spectrometry).
Step 7: Exclusion of highly hydrophobic epitopes to avoid peptide solubility problems during vaccine formulation
   Exclude peptides with more than 64% hydrophobic amino acids
Step 8: Exclusion of epitopes with certain problematic amino acid motifs, such as, for example:
   more than one cysteine (C) which are prone to oxidation and which can lead to intra- and inter-molecular disulfide-bridge formation and peptide complexation
   glutamine (Q) or glutamate (E) at the N-terminus which can spontaneously cyclize to pyroglutamate
   certain poly-amino acid-stretches (>=NNN) equal or longer than 3 amino acids
Step 9: Determination of loss of HLA alleles in the tumor with respect to the normal tissue tested in step 3. For example, by
   Determination of HLA class I and/or class II status (mutation or deletion) in the tumor tissue using tumor exome data or immunohistochemistry
   Determination of beta-2 microglobulin status in the tumor tissue using tumor exome data or immunohistochemistry. If B2M is mutated or lost, the HLA class I complex cannot be formed on the tumor cell surface and no class I restricted peptides can be presented on tumor cells.
   If available, expression of HLA molecules and B2M can be confirmed in the tumor transcriptome data
Step 10: Exclusion of epitopes predicted to bind only to HLA molecules which are lost in the tumor (as determined in step 9)
Step 11: Independent prioritization of neoantigens potentially binding to either class I or class II HLA molecules of the patient to identify optimal candidates for vaccination. A scoring scheme for either short HLA class I restricted epitopes or long class II restricted epitopes should include one or more of the following steps:
   Prioritization of epitopes from known cancer-related genes (CeGaT tumor panel TUM01, 710 genes)
   Prioritization of epitopes which harbor variants with high allele frequencies (VAFs) in the tumor. Such variants are more likely present and translated in a high proportion of tumor cells. Prioritization of epitopes harboring variants with a high expression level in the tumor. This can be determined if e.g. tumor transcriptome data are available.
   Prioritization of epitopes with a high predicted binding affinity to the patient's HLA molecules
   Prioritizization of mutated epitopes with a stronger predicted HLA binding affinity than the corresponding wildtype epitope
   Prioritization of epitopes which are predicted to bind to more than one HLA allele
   Prioritization of epitopes which are predicted by more than one algorithm to bind to patient's HLA molecules
Step 12: Selection of a number of potential neoantigens for the design of a cancer vaccine
   Mutated epitopes with the highest score are selected in order to cover different variants (driver and drug resistance mutations favored) and if possible all HLA class I and/or class II alleles present and intact in the tumor.
   The presence of the respective DNA variant can be manually verified in the tumor exome data, in particular with computer support (e.g. by visual inspection of the NGS data using the Integrative Genomics Viewer) or with orthogonal methods like tumor transcriptome analysis, qRT-PCR, qPCR, dPCR or Sanger sequencing.
Step 13: Synthesis of the neoantigens selected in step 12 as e.g. mutated peptides
Step 14: Preparation of patient-specific neoantigen-targeting peptide vaccine, for example by:
   Solubilization of single peptides in DMSO
   Addition of water and pooling of all peptides (final DMSO conc.=10%; 400 μg each peptide/500 μl injection aliquot).
   Sterile filtration and filling up of vaccine aliquots in ready-to-use sterile empty glass vials
Step 15: Administration of the patient-specific neoantigen-targeting vaccine The vaccine is repeatedly injected intradermally together with one or more immune stimulating adjuvants.

### Example 2—Exemplary Method Outline for Selection of Predicted HLA-Class I Restricted Neoantigens With Expression Data

### 1. Input

1.1. Exome and transcriptome sequencing
   Somatic missense variants from the tumor/normal exome analysis (non-synonymous single and multi-nucleotide variants, Indels, gene fusions)
   corresponding tumor transcriptome data,.
   Patient's HLA genotype (determined, for instance, from exome data of the patient's normal sample)
1.2. Epitope generation and prediction of HLA binding affinities
   Definition of all possible 8-11 amino acid long mutated peptides which can be derived from the tumor-specific mutations.
   Prediction of HLA class I binding affinities for each mutated peptide and its wildtype counterpart using methods SYFPEITHI, netMHC, netMHCpan

### 2. Filtering

2.1. Filtering of potential neoantigens according to the predicted HLA class I binding affinity:
   Exclude neoantigens with affinity>500 nM (netMHC, netMHCpan) and <50% of max. score (SYFPEITHI)
2.2. Filtering of self-peptides.
   Exclude potential neoantigens with homology to any human wildtype sequence (UniProtKB/Swiss-Prot HUMAN.fasta.gz)
2.3. Expression data
   keep neoantigen if variant allele frequency (VAF)>=5% AND sequence coverage>=20
2.4. Neoantigen sequence parameters
   keep if content of hydrophobic AA<=64%.
   If gene is in CeGaT “TUM01” list of known tumor-related genes, keep if number of Cysteines<=1.
   If gene is not in CeGaT “TUM01” list of known tumor-related genes, keep if number of Cysteines=0
   Keep if poly-amino acid stretches<3 (remove e.g. QQQ)
2.5. HLA loss.
   HLA typing of tumor transcriptome, tumor exome and blood exome
   Loss of HLA locus or HLA expression (HLA-A, HLA-B, HLA-C on chr 6, B2M on chr 15) in the tumor has to be evaluated (CNV calls and allele frequencies in exome sequencing data). If certain HLA alleles are lost, mutated or not expressed in the tumorthose neoantigens exclusively predicted to bind such alleles have to be removed.

### 3. Scoring

3.1. Cancer-related gene (CeGaT tumor panel TUM01, 710 genes).
   Mutations of unknown consequence in any cancer-related gene from the TUM01 panel (SCORE 50)
3.2. Variant allele frequency (VAF).
   Define tumor content Y by histopathological evaluation or based on allele frequencies of detected somatic SNVs
   High variant allele frequency: VAF>=2/3*Y/2 (SCORE 45)
   medium variant allele frequency: 1/3*Y/2<=VAF<2/3*Y/2 (SCORE 20)
   low variant allele frequency: 0<VAF<1/3*Y/2 (SCORE 5)
3.3. HLA binding affinity
   The affinity score is calculated for each possible peptide/HLA pair on the original results of NetMHC, NetMHCpan, and SYFPEITHI. The affinity score for each peptide/HLA pair is calculated for each algorithm as described below and averaged.
   High affinity (a): a<=50 nm for netMHCpan and netMHC; a>=75% of max. score for SYFPEITHI (SCORE 40)
   Medium affinity (a): 50 nM<a<=200 nM for netMHCpan and netMHC; 60%<=a<75% of max. Score SYFPEITHI (SCORE 20)
   Low affinity (a): 200 nM<a<=500 nM for netMHCpan and netMHC; 50%<=a<60% of max. score SYFPEITHI (SCORE 10)
3.4. Variant expression level (in tumor transcriptome)
   variant allele frequency in RNA * transcripts per million (RNA VAF * FPKM)
   Rank according to (RNA VAF * FPKM). Exclude all with value 0. Count #of remaining variants.
   Level size (Is)=#of remaining variants/3
   High expression range: top ranked variant until top ranked-1*Is (SCORE 10)
   Medium expression range: top ranked—1*Is+1 until top ranked—2*Is (SCORE 5)
   Low expression range: remaining variants (SCORE 0)
3.5. HLA binding affinity mutated peptide vs wild-type peptide.
   The relative HLA binding score is calculated on the original results of NetMHC, NetMHCpan, and SYFPEITHI for the wildtype peptide (WT) and the mutated peptide (MUT) as shown below. The affinity score is calculated for each algorithm and averaged.
For SYFPEITHI (affinity is given in % of maximal possible binding, higher is better):
   Higher: MUT/WT>1.1 (SCORE 10)
   Equal: 0.9≤ MUT/WT≤1.1 (SCORE 0)
   Lower: MUT/WT<0.9 (SCORE-10)
For NetMHC and NetMHCpan (affinity is given in nM, small is better):
   Higher: MUT/WT<0.9 (SCORE 10)
   Equal: 0.9≤ MUT/WT ≤1.1 (SCORE 0)
   Lower: MUT/WT>1.1 (SCORE −10)
3.6. HLA promiscuity.
   For each peptide the number of different HLA alleles (HLA) are determined for which binding was predicted by any algorithm
   High: HLA≥3 (SCORE 10 )
   Medium: HLA=2 (SCORE 5)
   Low: HLA=1 (SCORE 0)
3.7. Prediction method congruence
   For each peptide/HLA pair the number of methods (m) is determined with which binding was predicted
   High: m=3 (SCORE 5)
   Medium: m=2 (SCORE 2.5)
   Low: m=1 (SCORE 0)

### 4. Calculation of Combined Score, Ranking, and Selection

4.1. For each peptide/HLA pair compute total score by adding individual scores from previous step.
4.2. Sort peptides according to total score.
4.3. Select top 20 ranked peptides and all peptides that are equally ranked to peptide 20 for each HLA allele and summarize in one list.
4.4. Sort by (in this order): Gene, Total Score, HLA Type
4.5. Mark with Flag 1: Peptide with highest Total Score for each gene. If two peptides for the same gene have equal score, mark both with flag 1
4.6. Sort by (in this order): Flag 1, HLA Type, Total Score
4.7. Mark top 4 peptides in “flag 1” list of each HLA allele with flag 2. If two have equal Total Scores, mark both with flag 2. If an HLA allele is underrepresented (having not 4 peptides with flag 1), add best scored peptides from peptides not marked with flag 1. If patient does not have six different HLA alleles, Mark for each HLA allele 20/number of HLA alleles (rounded up) with flag 2
4.8. Visually inspect sequencing data for all variants of flag 2 marked peptides
4.9. Select e.g. 7 peptides for synthesis: Best scored peptide for each HLA class I allele. Fill up with the best scored peptides for different alleles, starting with HLA-A or B alleles.
4.10. In case of any ambiguity follow rules below:
From two equally ranked peptides for different variants, but same HLA allele:
   1. Choose peptide with higher expression
   2. Choose peptide with higher affinity (original value)
   3. Choose peptide with higher promiscuity
   4. Choose peptide with lower hydrophobicity
From two equally ranked peptides for the same variant, but different HLA alleles:
   1. Choose peptide with underrepresented HLA type
   2. Choose peptide with higher affinity (original value)
   3. Choose peptide with higher promiscuity
   4. Choose peptide with lower hydrophobicity

### Example 3—Exemplary Method Outline for Selection of Manually Designed HLA-Class II Restricted Neoantigens Without Expression Data

### 1. Input

1.1. Exome sequencing
   Somatic missense variants from the tumor/normal exome analysis (non-synonymous SNVs, MNVs, Indels, gene fusions)
1.2. Epitope generation
Manually design class Il restricted peptides of 16 to 17 amino acids (AA). If possible place the altered amino acid/s in the center of the peptide and use the following rules:
   Missense SNVs: 8+1+8 32 17 AA
   In-frame Insertions/MNVs (of AA size x): 8−(x/2 rounded down)+x+8−(x/2 rounded down)=overall length 16 AA if x is even or=17 AA if x is odd.
   In-frame Deletions: choose 8 AA upstream and 8 AA downstream of deletion;
   Indels leading to frameshift mutations: choose 8 AA upstream and 8 AA downstream of the frame shift start
   Gene fusions: choose 8 AA upstream and 8 AA downstream of breaking point; if protein sequence of either site is <8 than add missing AA on the other side so total peptide length is 16 AA.
   For any of above variants, except of in-frame insertions/MNVs: if variant is near to either protein end and hence protein sequence at either side of the variant is <8 AA then add missing AA on the other side so total peptide length is always at least 16 AA.
   For in-frame insertions/MNVs (of size x amino acids): if altered amino acides are near to either protein end and hence protein sequence at either side of the variant is <8 AA−(x/2 rounded down) then add missing AA on the other side so total peptide length is 16 AA if x is even or 17 AA if x is odd.

### 2. Filtering

2.1. Filtering of self-peptides
   Exclude potential neoantigens with homology to any human wildtype sequence (UniProtKB/Swiss-Prot HUMAN.fasta.gz)
2.2. Gene expression estimate
Check expression of protein (alternatively RNA) by database search for respective tumor type (The Human Protein Atlas, if not available, GEO). Exclude peptides of genes that are not expressed in tumor type.
2.3. Neoantigen sequence parameters
   keep if % hydrophobic AA<=64.
   If gene is in CeGaT “TUM01” list of known tumor genes, keep if number of Cysteines<=1.
   If gene is not in CeGaT “TUM01” list of known tumor genes, keep if number of Cysteines=0
   Keep if poly-amino acid stretches <3 (remove e.g. QQQ)

### 3. Scoring

3.1. Cancer gene (CeGaT TUM01, 649 genes)
   Mutations of unknown consequence in any cancer-related gene listed in CeGaT's tumor panel TUM01 (SCORE 50)
3.2. Variant allele frequency (VAF).
   Define tumor content Y
   High variant allele frequency: VAF>=2/3*Y/2 (SCORE 45)
   medium variant allele frequency: 1/3*Y/2<=VAF<2/3*Y/2 (SCORE 20).
   low variant allele frequency: 0<VAF<1/3*Y/2 (SCORE 5)
3.3. Gene expression estimate
Check expression of protein by database search for respective tumor type (The Human Protein Atlas, if not available, GEO). Mark expression level in respective tumor tissue: high/medium/low/heterogenic. “High” is assigned SCORE 10, “Medium” is assigned SCORE 5, “Low” or “Heterogenic” is assigned SCORE 0.

### 4. Calculation of Combined Score, Ranking, and Selection

4.1. Compute for every potential class Il restricted neoantigen total score by adding individual scores from previous step
4.2. Sort peptides according to total score
4.3. Select e.g. top 3 peptides. In case of ambiguities follow the rules below:
From two equally ranked peptides harboring different variants:
   1. Choose peptide harboring variant with higher expression
   2. Choose peptide harboring variant with higher VAF
   3. Choose peptide with lower hydrophobicity
4.4. If HLA class II and class I restricted peptides should be combined in a vaccine (see example 2), exclude all HLA class II peptides harboring variants already covered by class I peptides.

### Example 4: Comparison of Peptide Ensembles Obtained According to Different Methods

As stated above, for treating a patient, it is typically useful and preferred to select more than one neoantigen. In case more than one neoantigen is selected, care can be taken to increase the likelihood that the selection is effective by requesting that the neoantigens selected together have certain properties as an ensemble. For example, care can be taken that different HLA molecules are considered.

However, when selecting a plurality of neoantigens such that the ensemble together has certain properties, care must be taken that the overall ensemble still has favorable properties. It will be understood that comparing the results obtained by different selection methods in a statistically relevant and thus very large number of patients is not an option ethically defensible nor is it feasible given that each tumor has different HLA type and mutations requiring a unique and personalized neoantigen selection for every patient. Hence results obtained for one patient cannot be compared with those of another patient. Therefore, the only valid comparison would be to test different neoantigen ensembles obtained by various selection approaches within one patient. But the effort, costs and burden for the patient to test several ensembles is too high to be justified. Therefore, the results obtained by different methods must be compared in a different manner.

To this end, based on data obtained from an actual cancer patient an ensemble of 5neoantigen-peptides was determined and the results thereof evaluated in view of averages of values of the ensemble. In particular, for each of the respective 5 peptides obtained by the different methods, allele frequency, a degree of promiscuity, binding affinity and difference between wildtype peptide and mutated peptide were compiled. Furthermore, it was indicated what gene the peptide belongs to, whether the gene was known to be cancer-related, and also the binding HLA allele was determined.

This compilation is then used to compare the quality of the different ensembles obtained with various selection methods.

a—Ensemble by Random Selection

In a first approach, five peptides were randomly selected from a list of peptides predicted to be neoantigens for a tumor.

For these 5 peptides, allele frequency, promiscuity, binding affinity and difference between wildtype peptide and mutated peptide were calculated. Furthermore, it was determined what gene the peptide belongs to, whether the gene was known to be cancer-related, and the binding HLA allele was determined.

The following results were obtained (Ensemble a):

| | | | | | Diff. | | | |
|---|---|---|---|---|---|---|---|---|
| | | VAF | Tumor | Affinity | Affinity | HLA | | |
| Peptide | Gene | (%) | gene | (nM) | M-WT (nM) | Promisc | Peptide | HLA allele |
| 1 | CNN2 | 6.8 | no | 64 | 5 | 1 | DPGEAPEY | HLA-B*35:01 |
| 2 | SFI1 | 5.2 | no | 177 | −213 | 1 | QLLYVQKGKQK | HLA-A*03:01 |
| 3 | TRAPPC8 | 5.4 | no | 175 | −391 | 1 | FTSRSLNV | HLA-C*05:01 |
| 4 | LONP1 | 12.5 | no | 138 | −91 | 1 | GFTLFVETSLR | HLA-A*31:01 |
| 5 | ALAS1 | 10.2 | no | 213 | −170 | 2 | RSDPSFPK | HLA-A*03:01 |

It was thus found that the mean allele frequency of the five peptides is rather low, having a value of about 8%. The mean binding affinity is 153, the mean difference between wildtype binding affinity and mutant binding affinity is a mere −172 nM. The ensemble covers four different HLA alleles but none of the peptides bind to more than one HLA allele and none relates to a tumor gene.

b-Ensemble According to Score of Unweighted Parameters

While a random selection of peptides is an extremely easy approach, it will be obvious to a skilled person that a variety of parameters may be considered to improve the selection. Accordingly, the random selection given above basically can serve as a base line.

If some general knowledge of topics such as tumor genetics, degradation of proteins in a cell, and the presentation of peptides at the cell surface is used, a number of parameters can be selected for establishing a score of peptides. Using such a score, five peptides can be selected that each relate to a different gene.

For this example, it is considered whether the neoantigen is known to reside within a cancer-related gene.

Then, an average skilled person might want to consider whether the difference between the HLA binding affinity of the (subject specific) potential neoantigen and the corresponding non-mutated wild-type is large or not; in other words, the relative HLA binding affinity of the potential neoantigen as compared to the corresponding non-mutated wild-type sequence may be considered.

Also, the binding affinity of the mutated peptide may be considered as obtained, using the values obtained both by NetMHC and NetMHCpan and averaging these values. Finally, the promiscuity is taken into account, i.e. the number of alleles a peptide can bind to.

In order to select five peptides based on these four parameters, an overall score must be determined. Here, it must be taken into account that the different parameters will have very different values. In order to determine an overall score, a simple approach is to rank the set of peptides with respect to each parameter, giving four rankings for each peptide considered and to then add all the rankings a peptide has obtained. An overall “score” is determined based on this sum, favoring those peptides having the lowest rank.

Using this sum, a selection of five peptides can then be made, taking care that any gene is selected only once. Accordingly, a peptide will be selected for the ensemble only if all higher ranked peptides selected relate to a different gene.

The following results were obtained (Ensemble b):

| | | | | | Diff. | | | |
|---|---|---|---|---|---|---|---|---|
| | | VAF | Tumor | Affinity | Affinity | _HLA | | |
| Peptide | Gene | (%) | gene | (nM) | M-WT (nM) | Promisc | Peptide | HLA allele |
| 1 | LONP1 | 12.5 | no | 134 | −7,442 | 1 | LAWTAMGGF | HLA-B*35:01 |
| 2 | MED16 | 25.5 | no | 70 | −10,565 | 1 | SPGDRLTEIY | HLA-B*35:01 |
| 3 | GBP4 | 10.9 | no | 56 | −17,150 | 2 | RSFQEYMAQMK | HLA-A*03:01 |
| 4 | PRR21 | 28.2 | no | 28 | 19 | 1 | SSTPLHPR | HLA-A*31:01 |
| 5 | PERM1 | 32.0 | no | 14 | 4 | 1 | RYFRRQAGQGR | HLA-A*31:01 |

It was thus found that for the five peptides suggested, a very high affinity with a mean value of 60 nM was achieved and that the mean difference between wildtype binding affinity and mutant binding affinity is −7,026 nM. The mean allele frequency of the five peptides is about 22%. No tumor genes have been selected.

### C-Ensemble According to Score of Parameters Weighted According to the Invention

While the approach under “b” is an improvement over a random selection, it will be understood that selecting peptides relating to tumor genes might improve the overall results. To evaluate whether this leads to any improvement, a method similar to “b” is executed, with the only difference that once the sum of the four rankings is obtained, first of all, peptides relating to tumor genes are selected. Only in case no further tumor gene related peptides are found may high ranking non-tumor gene related peptides be selected,

In this manner, the following selection has been made (Ensemble c):

| | | | | | Diff. | | | |
|---|---|---|---|---|---|---|---|---|
| | | VAF | Tumor | Affinity | Affinity | HLA | | |
| Peptide | Gene | (%) | gene | (nM) | M-WT (nM) | Promisc | Peptide | HLA allele |
| 1 | CHD4 | 10.9 | yes | 122 | −30,863 | 1 | VVMDLKKCR | HLA-A*31:01 |
| 2 | PIK3CA | 11.2 | yes | 111 | −12,291 | 1 | YFMKQMNDAR | HLA-A*31:01 |
| 3 | PARK2 | 6.5 | yes | 56 | −28 | 1 | RNDWTVQNF | HLA-C*04:01 |
| 4 | LONP1 | 12.5 | no | 134 | −3,119 | 1 | LAWTAMGGF | HLA-B*35:01 |
| 5 | MED16 | 25.5 | no | 70 | −9,466 | 1 | SPGDRLTEIY | HLA-B*35:01 |

As can be seen, the five peptides suggested have a mean affinity value of 71 nM, which is slightly higher than that obtained in method “b” and a larger difference of wild type and mutant binding affinities, the mean difference being −11,358 nM. The mean allele frequency of 13% is lower than in “b” and of the five peptides selected, three relate to tumor genes.

d-Ensemble Selection According to Invention

Considering that a selection based primarily on tumor genes may result in selection of peptides for an ensemble that might have a variety of disadvantageous properties, a scoring according to the invention is suggested such that inter alia, the overall score a peptide may obtain will not be solely dominated by whether or not the peptide is tumor gene related.

In this manner, it can e.g. be avoided that tumor gene related peptides having hardly usable binding affinities will be preferred over non-tumor gene related peptides.

The following results were obtained (Ensemble d):

| | | | | | Diff. | | | |
|---|---|---|---|---|---|---|---|---|
| | | VAF | Tumor | Affinity | Affinity | HLA | | |
| Peptide | Gene | (%) | gene | (nM) | M-WT (nM) | Promisc | Peptide | HLA allele |
| 1 | CHD4 | 10.9 | yes | 122 | −30,863 | 1 | VVMDLKKCR | HLA-A*31:01 |
| 2 | PIK3CA | 11.2 | yes | 129 | −16,807 | 1 | FMKQMNDAR | HLA-A*31:01 |
| 3 | GBP4 | 10.9 | no | 56 | −17,150 | 2 | RSFQEYMAQMK | HLA-A*03:01 |
| 4 | PARK2 | 6.5 | yes | 56 | −28 | 1 | RNDWTVQNF | HLA-C*04:01 |
| 5 | PERM1 | 32.0 | no | 14 | 4 | 1 | RYFRRQAGQGR | HLA-A*31:01 |

In the example given, it can be seen that non-tumor gene peptide in GBP4 has a better score than the lower ranked tumor-gene related peptide in PARK2. Furthermore, a peptide having a promiscuity of 2 suggested according to method “b”, but disregarded using method “c” is included in the ensemble.

The preferred method suggests five peptides having a mean affinity similar to method “c” (with a mean value of 75 nM), but showing a larger difference of wild type and mutant binding affinities, the mean difference being −12,969 nM. The average allele frequency is 14% and thus higher than in method “c”. As in method “c” three out of five peptides relate to tumor genes.

This shows that the method according to the invention using an improved score is giving results that improve on allele frequency and difference of wild type and mutant binding affinities while not affecting affinity itself.

The following comparison summarizes these findings indicating that for an overall ensemble obtained according to the method of the present invention, relevant properties are on average found to be very good. It can be appreciated that administering these peptides in a pharmaceutical composition will give very good results in treating a patient because the likelihood is reduced that all neoantigens will turn out to be ineffective for unknown, unpredicted or underestimated reasons. Also, when an HLA allele is lost in the course of the treatment due to immunogenic pressure, the preferred ensemble will contain further peptides targeting neoantigens which bind to different HLA alleles. Here, targeting a set of neoantigens binding to several HLA alleles reduces the impact of competition for binding to one certain HLA allele and immunodominance effects of one peptide over the others.

| | Avg. | Avg. | Avg. Diff. | | | |
|---|---|---|---|---|---|---|
| | VAF | Affinity | Affinity | Tumor | Avg. HLA | #Alleles |
| Method | (%) | (nM) | MUT-WT (nM) | Genes | Promiscuity | covered |
| a) random | 8.0 | 153.4 | −172 | 0 | 1.2 | 4 |
| b) unweighted | 21.8 | 60.2 | −7.027 | 0 | 1.2 | 3 |
| c) weighted | 13.3 | 71.1 | −11.358 | 3 | 1.0 | 3 |
| d) invention | 14.3 | 75.1 | −12.969 | 3 | 1.2 | 3 |

### Example 5—Vaccination Regime for Adult Cancer Patients

### Vaccine

Intra-dermal injections of formulated peptides (400 μg each/dose); short class I restricted peptides (8-11 amino acids) & long class Il restricted peptides (˜17 amino acids). Note that 400 μg per peptide and injection were used independent of the weight of a patient.

### Adjuvants

Subcutaneous injection of Leukine (GM-CSF)

### Administration

Day 1, 3, 8, 15, 29. Monthly repeats.

### Example 6—Personalized Neoantigen-Targeting Vaccines

The methods described above have been used to develop personalized neoantigen-based vaccines for the treatment of cancer patients. Each resulting vaccine consisted of up to 20 peptides resembling distinct non-self antigens derived from tumor-specific mutations (neoantigens), not present in the normal tissues of the respective patient. In order to elicit a sustained immune response against cancer cells presenting such neoantigens via MHC on their surface, a peptide vaccine was repeatedly applied together with an immunostimulatory adjuvant (Leukine, GM-CSF). According to the established vaccination schedule, the personalized peptide vaccine was injected intradermally in the upper thigh or abdomen on days 1, 3, 8, 5, 29 and subsequently every 4 weeks (0.4 mg each peptide/injection). In order to increase the immune response to the vaccinated peptides, the adjuvant Leukine (GM-CSF, 83 μg/injection) was additionally injected subcutaneously in close proximity to the vaccination site ( )

Each vaccination cocktail consisted of short peptides (8 to 11 amino acids) and long peptides (15 to 21 amino acids). While short peptides are taken up and presented by antigen presenting cells (APCs) via MHC I molecules in order to activate neoantigen-specific cytotoxic T cells (CD8+), long peptides are internalized, processed and presented by APCs via MHC II molecules in order to activate neoantigen-specific T-helper cells (CD4+). The aim was to activate both T-cell populations, as they are thought to play distinct but complementary roles in the fight against tumor cells (Braumuller, H.; Wieder, T.; Brenner, E.; Assmann, S.; Hahn, M.; Alkhaled, M. et al. (2013) T-helper-1-cell cytokines drive cancer into senescence in: Nature 494 (7437), S. 361-365. DOI: 10.1038/nature11824; Dudley, M. E.; Gross, C. A.; Langhan, M. M.; Garcia; Sherry, R. M.; Yang, J. C. et al. (2010): CD8+ enriched “young” tumor infiltrating lymphocytes can mediate regression of metastatic melanoma in: Clinical cancer research: an official journal of the American Association for Cancer Research 16 (24), S. 6122-6131. DOI: 10.1158/1078-0432.CCR-10-1297; Heemskerk, B.; Kvistborg, P.; Schumacher, T. N. (2013): The cancer antigenome in: The EMBO journal 32 (2), S. 194-203. DOI: 10.1038/emboj.2012.333; Kreiter, S.; Vormehr, M.; van de Roemer, N.; Diken, M.; Lower, M.; Diekmann, J. et al. (2015): Mutant MHC class Il epitopes drive therapeutic immune responses to cancer in: Nature 520 (7549), S. 692-696. DOI: 10.1038/nature14426;Schumacher, T. N.; Schreiber, R. D. (2015): Neoantigens in cancer immunotherapy in Science (New York, N.Y.) 348 (6230), S. 69-74. DOI: 10.1126/science.aaa4971; Tran, E.; Turcotte, S.; Gros, A.; Robbins, P. F.; Lu, Y. C.; Dudley, M. E. et al. (2014): Cancer immunotherapy based on mutation-specific CD4+ T cells in a patient with epithelial cancer in: Science (New York, N.Y.) 344 (6184), S. 641-645. DOI: 10.1126/science.1251102.).

A number of patients suffering from tumors of diverse origin and late stage, which were refractory to standard therapies, were treated on a compassionate-use basis with personalized neoantigen-targeting multi-peptide vaccines designed by the methods described in the invention. The use of the personalized vaccines was registered by the local authorities in Germany (Regierungspräsidium Tuebingen) and all German regulations for compassionate use treatment were followed. In general, the patients showed promising outcomes. The first patient, suffering from a pancreatic carcinoma, started with vaccinations 4.5 years ago and is still alive (Sonntag K., Hashimoto H., Eyrich M., Menzel M., Schubach M., Döcker D., Battke F., Courage C., Lambertz H., Handgretinger R., Biskup S., Schilbach K. Immune monitoring and TCR sequencing of CD4 T cells in a long term responsive patient with metastasized pancreatic ductal carcinoma treated with individualized, neoepitope-derived multipeptide vaccines: a case report in J Transl Med. 2018 Feb. 6;16(1):23. DOI: 10.1186/s12967-018-1382-1). For a total of 12 patients with various malignancies long-term follow-up data including immunogenicity data are shown in FIG. 1. Each patient received repeated vaccinations utilizing between 3 and 11 peptides for at least 2.5 months before vaccine specific T-cell responses were assessed by intracellular cytokine staining and FACS analysis. Vaccine-specific T-cell responses were detected in all of these patients, except for one (patient no 9). An immune response was detectable to 53% of vaccinated peptides (54/101). Several peptides elicited CD4+, as well as CD8+ T cell responses (14%). Overall, 48% of the vaccinated peptides were recognized by CD4+ and 20% by CD8+ T cells.

For nine patients, evaluable data from several subsequent time points were available, and for seven of those, immune responses increased in the course of the vaccination schedule (exemplified in FIG. 2).

Prior to vaccination one breast cancer patient (No. 2), displayed already existing T cell responses against five of 10 peptides included in the vaccination cocktail (3 CD8+ and 2 CD4+ T cell responses). Therefore, the in-silico predicted neoantigen-peptides of the vaccine must have been presented via MHC molecules on tumor cells in vivo and prior to vaccination. This, in turn, led to a naturally occurring and efficient priming of neoantigen-specific T cells (FIG. 3: exemplary immune response to peptide MSYQGLPSTQL, NOTCH1-p.R2372Q). These results highlight that indeed the selected neoantigens were presented on the tumor-cell surface and that the applied neoantigen prediction and selection procedure is capable of identifying such novel and immunogenic tumor-epitopes. As the described patient is currently in complete remission, it is tempting to speculate that the tumor-specific immune response may have contributed to the positive outcome. Furthermore, these findings affirm the conclusion that the induction of a neoantigen-specific immunity in patients, who have not established a natural immune response against the same tumor-antigens before, might be of high clinical relevance.

In summary, results from immune-monitoring experiments performed for 12 vaccinated cancer patients demonstrated that efficient neoantigen-specific T cell responses (CD4+ and CD8+) are elicited upon vaccine injection. Such immune responses were observed to continually increase during the treatment. Preexisting immune responses against vaccine peptides which were detected prior to the vaccination further indicated, that the respective neoantigens were presented to the immune cells on the tumor cell surface before vaccination and that the established neoantigen selection process of the invention leads to the efficient selection of such immunogenic tumor-specific epitopes.

From the above, it is obvious that the disclosure of the present invention also comprises inter alia a pharmaceutical composition prepared as suggested in either the claims and/or the description for use in treating cancer. What is also disclosed is the use of a neoantigen selected in accordance with a method according to any of the claims in preparing a personalized pharmaceutical composition. Furthermore, a method of treating cancer, comprising administering to a patient in need thereof an effective amount of a pharmaceutical composition as claimed is suggested.

1. A computer-assisted_method for generating an individual physical library of neoantigens based on one or more neoantigens present in a subject having cancer, wherein one or more potential neoantigens in a plurality of potential neoantigens carry at least one tumor-specific mutation considered to be cancer-specific, the method comprising:
generating for the subject, having cancer, a library of potential neoantigens;
determining, for each potential neoantigen in the library, the library including at least four potential neoantigens, at least two descriptors, the at least two descriptors include at least two of the following:
(i) an indicative descriptor indicating whether a potential neoantigen in the library is known to reside within a cancer-related gene or whether the potential neoantigen is not known to reside within a cancer-related gene;
(ii) a classifying descriptor relating to a binning of a value indicative for an allele frequency of at least one tumor-specific mutation in the potential neoantigen of the subject into one of at least three different classes ordered according to intervals of values binned into each class;
(iii) a classifying descriptor relating to a binning of a value indicative for an expression rate of at least one variant within a neoantigen in one or more cancerous cells of the subject into one of at least three different classes ordered according to the intervals of values binned into each class;
(iv) a classifying descriptor relating to a binning of a value indicative for a binding affinity of the potential neoantigen to a particular HLA allele present according to the subject's HLA type, into one of at least three different classes ordered according to the intervals of values binned into each class;
(v) a classifying descriptor relating to a binning of a value indicative for a relative HLA binding affinity of the subject-specific potential neoantigen as compared to a corresponding non-mutated wild-type sequence into one of at least three different classes ordered according to the intervals of values binned into each class;
(vi) a classifying descriptor relating to a binning of a value indicative for a binding affinity of the potential neoantigen to more than one HLA allele present according to the subject's HLA type, into one of at least three different classes ordered according to the intervals of values binned into each class;
(vii) a classifying descriptor relating to a binning of a value indicative for an HLA promiscuity of the potential neoantigen, meaning the number of HLA alleles the potential neoantigen may bind to, into one of at least three different classes ordered according to the intervals of values binned into each class;
(viii) a classifying descriptor relating to a binning of a value indicative for a reliability of predicting binding of the subject specific potential neoantigen to an HLA allele of the subject into one of at least three different classes ordered according to the intervals of values binned into each class;
wherein the determination of at least one of the at least two descriptors being such that a number of different classes into which respective values are binned is smaller than a number of the potential neoantigens of the library;
determining a combined score for each of the potential neoantigens in the library based on the at least two descriptors such that a maximum possible contribution of at least one descriptor in the at least two descriptors to the combined score is lower than a maximum possible contribution to the combined score of at least one other descriptor in the at least two descriptors;
ranking the at least four potential neoantigens in the library based on the combined scores;
filtering out potential neoantigens; and
generating, using the library and based on the determinations with respect to the at least one neoantigen, an individual physical library of potential neoantigens, the individual physical library by having: one or more peptides of at least one neoantigen in the remaining potential neoantigens, at least one or more nucleic acids encoding at least one neoantigen in the remaining potential neoantigens, at least one or more vectors encoding at least one neoantigen in the remaining potential neoantigens, and any combination thereof synthesized.

2. The method according to claim 1, wherein the combined score for each of the potential neoantigens in the library is determined in a manner weighted such that for at least one classifying descriptor, a class dependent contribution to the combined score will for at least one class deviate from a linear relation with class order or will be a penalty.

3. The method according to claim 1, wherein for at least two descriptors (a, b) contributing to a combined score S additively in a manner S32 S(a)+S(b), at least one pair of values (a1, a2) that a first descriptor in the at least two descriptors may take, and at least one pair of values (b1, b2) that a second descriptor in the at least two descriptors may take exist, such that the contribution S(a)+S(b) to the combined score is such that S(a1)+S(b1)>S(a2)+S(b1), S(a2)+S(b1)>S(a2)+S(b2) but S(a1)+S(b2)>S(a2)+S(b1).

4. The method according to claim 1, wherein the individual library of potential neoantigens is provided in response to exome and/or transcriptome sequencing of subject specific biological material.

5. The method according to claim 1, wherein the individual library of potential neoantigens is provided by somatic missense variant identification.

6. The method according to claim 1, wherein the individual library of potential neoantigens is provided by analyzing at least one of a fresh frozen tumor sample, formalin-fixed paraffin-embedded tumor material, a stabilized tumor sample, a tumor sample stabilized in PaxGene Tubes, circulating tumor DNA, or circulating/disseminated tumor cells.

7. The method according to claim 1, wherein the indicative descriptor indicating whether the neoantigen is known to reside within a cancer-related gene or whether the neoantigen is not known to reside within a cancer-related gene has a first value, if the neoantigen is known to be cancer-related and having one of at least two values different from each other and both different from the first value and depending on the likelihood the neoantigen is not cancer-related.

8. The method according to claim 1, further comprising filtering out potential neoantigens prior to a subsequent selection, or handicapping the combined score of potential neoantigens prior to ranking,
the handicapping or filtering out being based on at least one of:
a value relating to the HLA binding affinity of the neoantigen;
a value relating to the neoantigen peptide length;
a value relating to the neoantigen being a self-peptide;
a value relating to the neoantigen expression rate;
a value relating to the neoantigen hydrophobicity;
a value relating to the number of cysteine residues of the neoantigen;
a value relating to the neoantigen having an N-terminal glutamine or glutamate; and/or
a value relating to the neoantigen poly-amino acid stretches.

9. The method according to claim 1, further comprising
determining at least one classifying descriptor relating to the binning of a value,
determining at least one value subjected to binning to obtain at least one classifying descriptor,
wherein the individual library of potential neoantigens is generated in response to an analysis of at least one of the following: a biological sequence data, a DNA sequence data, an RNA sequence data, a protein sequence data, a peptide sequence data, such data obtained from one of subject-specific biological tumor material, such tumor material and additionally subject-specific biological non-tumor material, by high-throughput DNA sequencing of at least a number of genes, high-throughput sequencing of messenger RNA (mRNA) molecules or total RNA, and/or by protein or peptide sequence analysis using low-or high-throughput tandem mass spectrometry, by proteomics, HLA-ligandomics, and any combination thereof.

10. The method according to claim 1, wherein at least one of
a classifying descriptor relating to the binning of a value of a binding affinity to a particular HLA allele present according to the subject's HLA type, into one of at least three different classes ordered according to the intervals of values binned into each class;
a classifying descriptor relating to the binning of a value of a relative HLA binding affinity of the subject specific potential neoantigen as compared to the corresponding non-mutated wild-type sequence into one of at least three different classes ordered according to the intervals of values binned into each class;
a classifying descriptor relating to the binning of a value of a binding affinity to more than one HLA allele present according to the subject's HLA type, into one of at least three different classes ordered according to the intervals of values binned into each class;
a classifying descriptor relating to the binning of a value of an HLA promiscuity of a neoantigen into one of at least three different classes ordered according to the intervals of values binned into each class;
is determined and wherein for determination of the value classified, HLA alleles for which a lower concentration in tumor cells derived from said subject having cancer is detected or assumed are excluded and/or wherein HLA alleles which have been found to be mutated or deleted in the tumor are excluded.

11. The method of claim 10, wherein one or more neoantigens predicted to bind only to one or more of the subject's HLA alleles being either deleted, mutated and/or not expressed in the tumor of the patient, are excluded.

12. The method according to claim 1, wherein at least one classifying descriptor is binning the respective value into one of not more than five ordered classes, into not more than four ordered classes, or into one of three ordered classes, and wherein all classifying descriptors are binning the respective value into one of not more than five classes, into not more than four classes, or into one of three classes.

13. The method according to claim 1, wherein the maximum possible contribution to the combined score of the descriptor relating to indicating whether the neoantigen is known to be cancer-related or is not known to be cancer-related is larger than the maximum possible contribution to the combined score of any single of the descriptors relating to the allele frequency of the at least one tumor-specific mutation in the neoantigen, the binding affinity to a particular HLA allele present according to the subject's HLA type, the relative expression rate of the neoantigen in one or more cancerous cells of the subject, the relative HLA binding affinity of the subject specific potential neoantigen as compared to the corresponding non-mutated wild-type sequence, the binding affinity to more than one HLA allele present according to the subject's HLA type, the HLA promiscuity and the reliability of predicting HLA binding of the subject specific potential neoantigen.

14. The method according to claim 1, wherein the maximum possible contribution to the combined score of the descriptor relating to the allele frequency of the at least one tumor-specific mutation in the neoantigen is larger than the maximum possible contribution to the combined score of any single of the descriptors relating to the binding affinity to a particular HLA allele present according to the subject's HLA type, the relative expression rate of the neoantigen in one or more cancerous cells of the subject, the relative HLA binding affinity of the subject specific potential neoantigen as compared to the corresponding non-mutated wild-type sequence, the binding affinity to more than one HLA allele present according to the subject's HLA type, the HLA promiscuity, and the reliability of predicting binding of the subject specific potential neoantigen.

15. The method according to claim 1, wherein the maximum possible contribution to the combined score of the descriptor relating to the binding affinity to a particular HLA allele present according to the subject's HLA type is larger than the maximum possible contribution to the combined score of any single of the descriptors relating to the relative expression rate of the neoantigen in one or more cancerous cells of the subject, the relative HLA binding affinity of the subject specific potential neoantigen as compared to the corresponding non-mutated wild-type sequence, the binding affinity to more than one HLA allele present according to the subject's HLA type, the HLA promiscuity, and the reliability of predicting binding of the subject specific potential neoantigen.

16. The method according to claim 1, wherein the maximum possible contribution to the combined score of the descriptor relating to the relative expression rate of the neoantigen in one or more cancerous cells of the subject is larger than the maximum possible contribution to the combined score of any single of the descriptors relating to the relative HLA binding affinity of the subject specific potential neoantigen as compared to the corresponding non-mutated wild-type sequence, the binding affinity to more than one HLA allele present according to the subject's HLA type, the HLA promiscuity, and the reliability of predicting binding of the subject specific potential neoantigen.

17. The method according to claim 1, wherein the maximum possible contribution to the combined score of the descriptor relating to the relative HLA binding affinity of the subject specific potential neoantigen as compared to the corresponding non-mutated wild-type sequence is larger or equal to the maximum possible contribution to the combined score of any single of the descriptors relating to the binding affinity to more than one HLA allele present according to the subject's HLA type, the HLA promiscuity and the reliability of predicting binding of the subject specific potential neoantigen.

18. The method according to claim 1, wherein the maximum possible contribution to the combined score of the descriptor relating to the binding affinity to more than one HLA allele present according to the subject's HLA type is larger than the maximum possible contribution to the combined score of the descriptors relating to the HLA promiscuity and the reliability of predicting binding of the subject specific potential neoantigen.

19. The method according to claim 1, wherein the maximum possible contribution to the combined score of the descriptor relating to the HLA promiscuity is larger than the maximum possible contribution to the combined score of the descriptor relating to the reliability of predicting binding of the subject specific potential neoantigen.

20. The method according to claim 13 wherein each of the respective possible contributions to the combined score obeys the relations indicated.

21. The method according to claim 1, wherein the method further comprises a step of selecting one or more of the ranked neoantigens.

22. A selection method for cancer-specific neoantigens, wherein a ranking according to claim 1 is determined and at least one neoantigen and less than all neoantigens from the plurality of potential neoantigens in view of the ranking are selected to form an ensemble of neoantigens.

23. The method of claim 22, wherein the neoantigens are selected in view of their ranking such that for each of a plurality of the HLA alleles considered, at least the highest ranked neoantigen is selected.

24. The method of claim 22, wherein if the ensemble comprises more neoantigens than the most favorably ranked neoantigens, then further highly ranked neoantigens for different alleles are selected starting with HLA-A or B alleles; and if at least two such highly ranked neoantigens for the same variant, but different alleles starting with HLA-A or B alleles are equally ranked, then a neoantigen with an HLA type allele hitherto underrepresented in the ensemble is selected, and if at least two such neoantigens exist binding to no hitherto underrepresented HLA allele, then a neoantigen thereof with a higher HLA binding affinity is selected, preferably a higher binding affinity not according to the classifying descriptor but according to the original value classified; and if at least two such neoantigens having an equal HLA binding affinity exist, then the neoantigen thereof having a higher HLA promiscuity is selected and if at least two such neoantigens having an equal HLA promiscuity exist, then the neoantigen thereof having a lower hydrophobicity is selected; and
if at least two such highly ranked neoantigens for different variants, but the same HLA allele are equally ranked, then the neoantigen thereof having the higher expression is selected; and if at least two such neoantigens having an equal expression exist, then the neoantigen thereof with a higher HLA binding affinity is selected, preferably a higher binding affinity according to not the classifying descriptor, but according to the original value classified; and if at least two such neoantigens having an equal HLA binding affinity exist, then the neoantigen thereof having a higher HLA promiscuity is selected and if at least two such neoantigens having an equal HLA promiscuity exist, then the neoantigen thereof having a lower hydrophobicity is selected.

25. The method according to claim 1, wherein HLA alleles are considered to be subject to an HLA gene deletion or mutation or reduction in expression derived in view of a tumor transcriptome, a tumor exome or a blood exome and/or an immunohistochemistry staining of a tumor and/or normal tissue sample.

26. The method according to claim 21, wherein the ensemble comprises at least one HLA class I restricted neoantigen and one HLA class II restricted neoantigen.

27. An ensemble of one or more peptides resembling one or more of the neoantigens ranked/selected according to claim 1.

28. A nucleic acid encoding one or more of the neoantigens ranked/selected according to claim 1.

29. The nucleic acid of claim 28, wherein the nucleic acid is a DNA or RNA molecule.

30. A vector comprising the nucleic acid of claim 28.

31. Eukaryotic or prokaryotic cells, bacteria or fungi expressing the nucleic acid of claim 28 and/or the vector of claim 30.

32. A pharmaceutical composition comprising one or more of the neoantigen(s) ranked or selected according to claim 1, the neoantigens being encoded either by peptides as in claim 27, nucleic acids as in claim 28, a vector as in claim 30 and/or the neoantigens are expressed in eukaryotic or prokaryotic cells, bacteria or fungi as in claim 31.

33-35. (canceled).

36. A data carrier comprising data relatable to at least one individual patient having cancer, the data carrier carrying data relating to a plurality of potential neoantigens harboring at least one mutation considered to be specific to the cancer of the at least one individual patient in that for each of at least four potential antigens of this plurality of neoantigens at least two data points of the group (a) through (h) are provided, with the group (a) through (h) consisting of
(a) an indicative descriptor indicating whether the neoantigen is known to reside within a cancer-related gene or whether the neoantigen is not known to reside within a cancer-related gene and/or a value indicative for a likelihood estimate that the neoantigen is not cancer-related;
(b) a classifying descriptor relating to the binning of a value indicative for the allele frequency of the at least one tumor-specific mutation in the neoantigen of the subject into one of at least three different classes ordered according to the intervals of values binned into each class and/or a value indicative for the allele frequency of the at least one tumor-specific mutation in the neoantigen of the subject;
(c) a classifying descriptor relating to the binning of a value indicative for a relative expression rate of the at least one variant within a neoantigen in one or more cancerous cells of the subject into one of at least three different classes ordered according to the intervals of values binned into each class and/or a value indicative for a relative expression rate of the at least one variant within a neoantigen in one or more cancerous cells of the subject;
(d) a classifying descriptor relating to the binning of a value indicative for a binding affinity of a neoantigen to a particular HLA allele present according to the subject's HLA type, into one of at least two different classes ordered according to the intervals of values binned into each class and/or a value indicative for a binding affinity of a neoantigen to a particular HLA allele present according to the subject's HLA type;
(e) a classifying descriptor relating to the binning of a value indicative for a relative HLA binding affinity of the subject specific potential neoantigen as compared to the corresponding non-mutated wild-type sequence into one of at least three different classes ordered according to the intervals of values binned into each class and/or a value indicative for a relative HLA binding affinity of the subject specific potential neoantigen as compared to the corresponding non-mutated wild-type sequence;
(f) a classifying descriptor relating to the binning of a value indicative for a binding affinity to more than one HLA allele present according to the subject's HLA type, into one of at least three different classes ordered according to the intervals of values binned into each class and/or a value indicative for a binding affinity to more than one HLA allele present according to the subject's HLA type;
(g) a classifying descriptor relating to the binning of a value indicative for the HLA promiscuity of a neoantigen into one of at least three different classes ordered according to the intervals of values binned into each class and/or a value indicative for the HLA promiscuity of a neoantigen;
(h) a classifying descriptor relating to the binning of a value indicative for the reliability of predicting binding of the subject specific potential neoantigen to an HLA allele of the respective patient into one of at least three different classes ordered according to the intervals of values binned into each class and/or a value indicative for the reliability of predicting binding of the subject specific potential neoantigen to an HLA allele of the respective patient; and/or the data carrier carrying data relating to neoantigen scoring as obtained by the method of claim 1; and/or the data carrier carrying data relating to one or more neoantigens selected according to the method of claim 1; and/or the data carrier carrying data relating to instructions to produce a pharmaceutical composition comprising at least one substance determined in response to a result of the method according to claim 1.

37. A kit comprising at least one of a container for biological material prepared in a manner allowing determination of personalized data usable as input into a method according to claim 1, wherein said biological material is obtained from a patient having cancer; or a data carrier storing personalized genetic data usable as individual-related input into said method and an information carrier carrying information relating to the identification of the patient; and instructions to execute said method and/or to provide data for the production of a data carrier as defined in the previous claim and/or to provide a data carrier.

38. . . . A biomarker for the diagnosis, monitoring and/or prognosis of tumor diseases, wherein the biomarker comprises one or more of the neoantigen(s) identified, ranked and/or selected by the method of claim 1.


SEQUENCE LISTING (21 sequences)

SEQ ID NO: 1 - protein, 8 aa, Homo sapiens
DPGEAPEY

SEQ ID NO: 2 - protein, 11 aa, Homo sapiens
QLLYVQKGKQK

SEQ ID NO: 3 - protein, 8 aa, Homo sapiens
FTSRSLNV

SEQ ID NO: 4 - protein, 11 aa, Homo sapiens
GFTLFVETSLR

SEQ ID NO: 5 - protein, 8 aa, Homo sapiens
RSDPSFPK

SEQ ID NO: 6 - protein, 9 aa, Homo sapiens
LAWTAMGGF

SEQ ID NO: 7 - protein, 10 aa, Homo sapiens
SPGDRLTEIY

SEQ ID NO: 8 - protein, 11 aa, Homo sapiens
RSFQEYMAQMK

SEQ ID NO: 9 - protein, 8 aa, Homo sapiens
SSTPLHPR

SEQ ID NO: 10 - protein, 11 aa, Homo sapiens
RYFRRQAGQGR

SEQ ID NO: 11 - protein, 9 aa, Homo sapiens
VVMDLKKCR

SEQ ID NO: 12 - protein, 10 aa, Homo sapiens
YFMKQMNDAR

SEQ ID NO: 13 - protein, 9 aa, Homo sapiens
RNDWTVQNF

SEQ ID NO: 14 - protein, 9 aa, Homo sapiens
LAWTAMGGF

SEQ ID NO: 15 - protein, 10 aa, Homo sapiens
SPGDRLTEIY

SEQ ID NO: 16 - protein, 9 aa, Homo sapiens
VVMDLKKCR

SEQ ID NO: 17 - protein, 9 aa, Homo sapiens
FMKQMNDAR

SEQ ID NO: 18 - protein, 11 aa, Homo sapiens
RSFQEYMAQMK

SEQ ID NO: 19 - protein, 9 aa, Homo sapiens
RNDWTVQNF

SEQ ID NO: 20 - protein, 11 aa, Homo sapiens
RYFRRQAGQGR

SEQ ID NO: 21 - protein, 11 aa, Homo sapiens
MSYQGLPSTQL